(19) United States
(12) Patent Application Publication
(10) Pub. No.: US 2024/0108598 A1
(43) Pub. Date: Apr. 4, 2024

(54) Danegaptide for Use in the Treatment or Prevention of a Kidney Disease

(71) Applicant: CIANA THERAPEUTICS APS, Rungsted Kyst (DK)

(72) Inventors: Paul Squires, Lincolnshire (GB); Claire Hills, Lincolnshire (GB); Ulrik Mouritzen, Rungsted Kyst (DK)

(73) Assignee: Breye Therapeutics ApS, Hørsholm (DK)

(21) Appl. No.: 18/275,254

(22) PCT Filed: Feb. 3, 2022
(86) PCT No.: PCT/EP2022/052649

§ 371 Date: Aug. 1, 2023

(30) Foreign Application Priority Data
Feb. 10, 2021 (EP) 21156236.8

(51) Int. Cl. A61K 31/401; A61P 13/12; G01N 33/50

(52) U.S. Cl. CPC A61K 31/401; A61P 13/12; G01N 33/5005; G01N 2800/347

(57) ABSTRACT

The present invention relates to danegaptide, or a pharmaceutically acceptable salt thereof for use in the treatment of a kidney disease, such as a chronic kidney disease, for example Diabetic Nephropathy and/or Chronic Kidney Disease.

### FIELD OF THE INVENTION

The present invention relates to danegaptide and its use in the treatment or prevention of a kidney disease, in particular kidney disease presenting with renal inflammation and/or renal fibrosis. The present invention further relates to treatment or prevention of a chronic kidney disease, for example diabetic nephropathy and/or chronic kidney disease, or kidney disease that results from another condition.

### BACKGROUND OF THE INVENTION

Chronic kidney disease (CKD) is the most common kidney disease, which represents a growing health concern associated with increased risk of cardiovascular disease, risk of developing end stage renal disease (ESRD) and increased morbidity and mortality. Estimated to affect 10% of the global population, risk factors include age, diabetes, hypertension, dyslipidaemia and obesity. The disease is characterised by a decline in glomerular filtration rate (GFR), hypertension and proteinuria, with glomerulosclerosis, tubular atrophy and tubulointerstitial fibrosis (TIF) observed as common histopathological changes. Culminating in the loss of kidney function, persistent inflammation and increased deposition of extracellular matrix, treatment and prevention of TIF and advanced CKD represents unmet clinical needs. Consequently, new therapeutic approaches are required.

Altered connexin (Cx) expression and function has been implicated in the pathology of various forms of kidney diseases, including CKD. Connexins are a family of membrane-bound proteins that oligomerise into hexameric assemblies termed connexons. The connexons, either function as a conduit for paracrine signalling and autocrine signalling, forming a transmembrane hemi-channel, or, if aligned with connexons on neighbouring cells, form a continuous aqueous pore or gap junction, which allows for the direct transmission of metabolic, cell signalling and electrical signals. In addition, connexins can play an important role in regulating cellular adhesion and interact directly adherens and tight junction complexes in a phosphorylation-dependent manner. Connexin 43 (Cx43) is one of the most abundantly expressed connexins in the renal vasculature and in the renal tubules. The cross-talk between adjacent cells via gap junctions is referred to as “gap junctional intercellular communication” (GJIC). Additionally, connexons, can function as hemi-channels also involved in communication between neighbouring cells, e.g. via release of ATP through the formed hemi-channels.

The ability of cells to communicate and synchronise their activity is in part involved in the maintenance of tissue structure, integrity and function. Regulation of connexin synthesis and activity is critical to cellular function, and a number of kidney diseases are attributed to changes in the expression and/or function of these important proteins.

In the kidneys of patients with diabetes mellitus, glycaemic injury is the leading cause of ESRD and kidney failure, reflecting multiple pathological events including glomerular hyper-filtration, albuminuria, increased deposition of extracellular matrix and TIF. Loss of connexin mediated cell-to-cell communication in diabetic nephropathy may represent an early sign of disease progression and glucose-evoked changes in connexin-mediated cell communication and associated purinergic signaling, such as P2X7, may further contribute to the pathogenesis of kidney disease in diabetes.

Danegaptide ((2S,4R)-1-(2-aminoacetyl)-4-benzamidopyrrolidine2-carboxylic acid), also known as ZP1609 or GAP-134, is a small dipeptide derived from rotigaptide (ZP123), Anti Arrhythmic Peptide (AAP) and AAP10. It was originally developed as a connexin 43 (Cx43) gap junction modifier and antiarrhythmic agent, and it has demonstrated cell protective and anti-arrhythmic properties (see WO 2007/078990 and WO 2008/079266).

The role of gap junction intercellular communication (GJIC) and connexin based hemi-channel signalling is complex. Several research groups have attempted to evaluate the relationship between Cx43 protein expression, phosphorylation status and protein function within different tissue type and disease presentations, including renal tissues, diabetic retinopathy, liver fibrosis and chronic and acute heart disease models. Importantly, Cx43 expression in these models varies according to cell and tissue type, and there is considerable uncertainty for the role of Cx43, which is upregulated in some stress-based models and downregulated in others. In particular for models of CKD, the stress induced changes in Cx43 expression are dependent upon the region of the kidney and the model of disease. For example, Cx43 exhibits increased expression in both glomeruli in patients with glomerular diseases and in biopsy material isolated from individuals with diabetic nephropathy. Further, in vivo studies using models of CKD suggest that Cx43 expression increases in the unilateral ureteral obstruction (UUO) model of advanced interstitial inflammation and fibrosis, the mouse model of glomerulonephritis (GN) and in the renin transgene (RenTG) mouse model of renin-dependent CKD. Implications of this increased expression have been evaluated using the heterogenous Cx43^{+/-} mouse model. When induced with glomerulonephritis, Cx43^{+/-} mice exhibit proteinuria, blood urea nitrogen (BUN) and serum creatinine levels significantly lower than those of wild-type animals. Moreover, heterogenous (Cx43^{+/-}) mice induced with unilateral ureteral obstruction (UUO) exhibit reduced extracellular matrix deposition and decreased inflammation as compared to WT control. Lastly, pharmacological studies using GAP-26 have shown that monocyte adhesion and expression of collagen I in the renin transgene (RenTG) mouse model of renin-dependent CKD is decreased when Cx43 activity is blocked. These data show that the response in kidney cells varies and that the changes in expression is not a universal response to injury in all kidney cell populations.

Contrary to the evidence presented above, Cx43 expression is reportedly down-regulated in the kidneys of db/db mice and in high glucose (HG)-induced rat proximal tubule cells. Furthermore, whilst podocytes exhibit increased expression of Cx43 in animals with glomerulonephritis and diabetic nephropathy, mesangial cells exhibit reduced expression of the same isoform when cultured in high glucose or aldosterone.

To date there is no clear evidence as to how altered Cx43 expression and function may optimally protect against the pathophysiological changes as observed in various tissues in different disease states. Importantly, whilst some studies have indicated that diminished Cx43 expression levels may confer protection against changes of CKD both in vitro and in vivo, it has not previously been investigated if danegaptide can protect against inflammation and fibrosis in the injured kidney. Hence, there is no clear consensus on how Cx43 ideally should be modified to ultimately provide therapeutic benefit for patients with kidney diseases.

### SUMMARY OF THE INVENTION

Broadly, the present invention is based on the new and distinct findings that the gap junction modulator danegaptide has the previously unknown effect of preventing Cx43 based hemi-channel release of ATP in treated and stimulated primary human proximal tubular epithelial cells. Blocking this ATP release was associated with a reduction in both key proinflammatory (e.g., Interleukin 6, Monocyte chemoattractant protein, MCP-1, RANTES), and profibrotic proteins (e.g., PDGF). Importantly, danegaptide also prevented the TGFβ-1 induced secretion of key chemokines known to recruit and activate neighbouring immune and extracellular matrix producing cells in the interstitium. Furthermore, the data presented in this application show that danegaptide protects against TGFβ1-induced cell-cell uncoupling through increased adherence, as evidenced by increased expression levels of adherence junctions and tight junctions. This improved coupling between cells was functionally associated with an improved barrier function as measured by transepithelial electrical resistance showing reduced paracellular leakiness under danegaptide treated conditions. The data disclosed herein highlights the therapeutic potential of targeting connexins with a specific gap junction modulator, as a novel method of treating kidney diseases, where inflammation and fibrosis represent the underlying pathology e.g. diabetic nephropathy and CKD. It further supports the claims of the present invention which are directed to the involvement of these processes in the treatment or prevention of kidney diseases, for example those diseases that present with renal inflammation and development of renal fibrosis, such as chronic kidney disease, typically in human patients.

Recently, the inventors have published a review of Cx43 as a target for treatment of inflammation in secondary complications of the kidney and eye in diabetes (Cliff et al., Int. J. Mol. Sci., 2022, 23, 600).

Based on these new findings, the present inventors have surprisingly found that danegaptide, or a pharmaceutically acceptable salt and/or hydrate thereof, is particularly suitable for treatment of kidney disease. Kidney diseases suitable for treatment with danegaptide may present with a range of renal dysfunction severities resulting from various renal pathologies, including but not limited to, renal inflammation and/or renal fibrosis. The data generated supports the observation that danegaptide works both to maintain the cell to cell coupling of human proximal tubular epithelial cells under stressed conditions and facilitates closure of hemi-channels, thereby limiting ATP leakage to the extracellular environment. The combination of these two effects provides for a novel and unique therapeutic potential of danegaptide, which is considered beneficial over other therapeutic interventions which only target hemi-channel closure.

The present inventors have found that danegaptide is able to negate the effects of altered Cx expression and function as observed in certain kidney diseases, such as diabetic nephropathy, glomerulonephritis and CKD. This results in restoration of changes in both expression and secretion of proteins linked to inflammation and fibrosis normally observed in kidney diseases such as CKD. Together the findings herein underline that danegaptide or a pharmaceutically acceptable salt and/or hydrate thereof can be used in the treatment of kidney diseases. In one embodiment, danegaptide hydrochloride can be used in the treatment of Chronic Kidney Disease (CKD).

Accordingly, in one aspect the present invention provides a compound of formula (I):

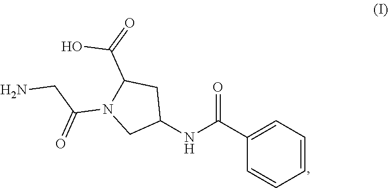 [SMILES: NCC(=O)N1CC(NC(=O)c2ccccc2)CC1C(=O)O]

or a pharmaceutically acceptable salt and/or hydrate thereof; for use in a method of treating or preventing a kidney disease. Preferably, the kidney disease is Chronic Kidney Disease (CKD) or an underlying condition leading to Chronic Kidney Disease (CKD).

In a further aspect, the present invention provides a method for selecting a subject for treatment by a compound of the present disclosure, the method comprising analysing a sample obtained from the subject for one or more a kidney disease biomarkers and, where the biomarker is present the step of selecting the subject for treatment. Examples of biomarkers for this purpose include, but are not limited to proteinuria, GFR estimations, urine albumin-to-creatinine ratio and/or biomarkers of inflammation or fibrosis development. Preferably, the method further comprises treating the subject using by a compound as defined herein.

In a further aspect, the present invention provides a method for treatment of a kidney disease in a subject, the method comprising administering to the subject a compound of formula (I):

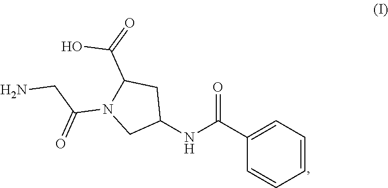 [SMILES: NCC(=O)N1CC(NC(=O)c2ccccc2)CC1C(=O)O]

or a pharmaceutically acceptable and/or hydrate salt thereof. Preferably, the kidney disease is Chronic Kidney Disease (CKD) or an underlying condition leading to Chronic Kidney Disease (CKD).

In a further aspect, the present invention provides the use of a compound of formula (I):

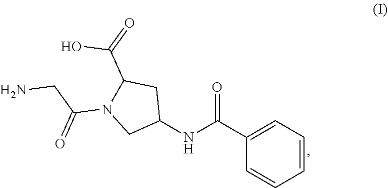 [SMILES: NCC(=O)N1CC(NC(=O)c2ccccc2)CC1C(=O)O]

or a pharmaceutically acceptable salt and/or hydrate thereof, for the preparation of a medicament for the treatment of a kidney disease in a human subject, the method comprising administering to the subject a therapeutically effective amount of the compound, or a pharmaceutically acceptable salt or hydrate thereof. Preferably, the kidney disease is Chronic Kidney Disease (CKD) or an underlying condition leading to Chronic Kidney Disease (CKD).
“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Unless context dictates otherwise, the descriptions and definitions of the features set out above are not limited to any particular aspect or embodiment of the invention and apply equally to all aspects and embodiments which are described.

All documents mentioned herein are expressly incorporated by reference in their entirety.

Embodiments of the present invention will now be described by way of example and not limitation with reference to the accompanying figures. However, various further aspects and embodiments of the present invention will be apparent to those skilled in the art in view of the present disclosure.

### BRIEF DESCRIPTION OF DRAWINGS

FIG. 1: Danegaptide prevents TGFβ1-evoked increases in hemichannel mediated dye uptake.

In panel A, HK2 cells were cultured in low glucose (5 mM) with/without TGFβ1 (10 ng/ml)±danegaptide (50, 100 and 1000 nM) for 48 h and cell viability assessed. Results are presented as mean±SEM, n=3. Incubation with TGFβ1 (10 ng/ml±danegaptide (50-1000 nM) did not alter MTT uptake, LDH release or crystal violet staining. In panel B and C, carboxyfluorescein dye uptake was used to assess hemichannel activity with the degree of dye loading being directly proportional to opening. Cells were cultured in low (5 mM) glucose with/without TGFβ1 (10 ng/ml)±danegaptide (50 or 100 nM) for 48 h. Danegaptide prevented TGFβ1-evoked increases in carboxyfluorescein dye uptake in HK2 (B) and hPTEC (C) cells. A dose range of 10 to 1000 nM danegaptide was tested and it was found that optimal inhibition of carboxyfluorescein dye uptake in HK2 Cells occurred at a concentration of 50-100 nM danegaptide (D). Minimal dye loading occurred in control cells, whilst dye loading significantly increased in cells treated with TGFβ1. Addition of danegaptide (50 or 100 nM) reduced dye uptake, returning fluorescence intensity to control levels. Intensity is expressed as percentage compared to low glucose control and is representative of 3 separate experiments. Data is presented as mean±SEM, n=3 with key significances indicated (**P<0.01, ***P<0.001); one-way ANOVA and Tukey's post-test).

FIG. 2: Danegaptide prevents TGFβ1-evoked increases in ATP release.

HK2 cells were cultured in low (5 mM) glucose with/without TGFβ1 (10 ng/ml)±danegaptide (50 or 100 nM) for 48 h. Representative biosensor traces show ATP release following removal of extracellular calcium. Control cells (A) exhibit negligible ATP release compared to a calibration (CALIB) response to 10 μM ATP, whilst a marked increase in release was observed from TGFβ1-treated cells (B). Danegaptide (100 nM) alone failed to alter basal ATP (C) but significantly reduced TGFβ1-evoked ATP release (D). Peak responses were quantified by comparing against a known concentration of ATP (10 μM) and mean data±SEM plotted (E). Results are representative of 3 separate experiments (n=3; **P<0.01, 777 ***P<0.001; one-way ANOVA and Tukey's post-test).

FIG. 3: Danegaptide reduces TGFβ1-induced mRNA changes in cell cycle and reno-protective markers.

Primary hPTECs were cultured in low (5 mM) glucose±TGFβ1 (10 ng/mL)±danegaptide (100 nM) for 12 h. In TGFβ1-treated cells, qPCR analysis demonstrated an increase in p16, p21 and cyclinD1 mRNA, and a significant reduction of Klotho mRNA as compared to control (**P<0.01 and ***P<0.001; mean±SEM, n=3). Danegaptide (100 nM) negated the increase in p16, p21, cyclinD1 (▴▴▴P<0.001; mean±SEM, n=3) and partly reversed the TGFβ1-evoked change in Klotho.

FIG. 4: Danegaptide reduces TGFβ1-evoked changes in adherens and tight junction proteins and epithelial leakiness.

Primary hPTECs were cultured in low (5 mM) glucose±TGFβ1 (10 ng/mL)±danegaptide (100 nM) for 48 h. Expression of E-cadherin (ECAD), N-cadherin (NCAD) and vimentin (panel A), and Claudin-2 and Zonula Occludens (ZO-1) and b-catenin (panel B) were assessed via western blotting. TGFβ1 reduced E-cadherin, Claudin-2 and ZO-1 expression and increased N-cadherin and vimentin expression (**P<0.01, ***P<0.001; mean±SEM, n=3). Effects were partially or completely reversed by danegaptide (100 nM; ▴P<0.05 and ▴▴▴P<0.001; mean±SEM, n=3). Representative blots for each protein are shown, with expression normalised by re-probing for α-tubulin as a loading control. In panel C, transepithelial electrical resistance (TER) assessed the consequence of altered adherens & tight-junction protein expression on epithelial integrity. HK2 cells were cultured in low (5 mM) glucose on transwell inserts and transepithelial resistance measured. TGFβ1 reduced TER, an effect partially restored by the addition of danegaptide (100 nM), data expressed as mean±SEM, n=3 (***P<0.001)

FIG. 5: Danegaptide negates TGFβ1-evoked changes in expression of proteins associated with the extracellular matrix (ECM).

Primary hPTECs were cultured in low (5 mM) glucose±TGFβ1 (10 ng/mL)±danegaptide (100 nM) for 48 h. In panel A, expression of the ECM proteins collagen I (Col1), collagen IV (Col4), fibronectin (Fibro), and Laminin (panel B) were assessed via western blotting. In all cases, TGFβ1 upregulated protein expression (***P<0.001), an effect reduced by danegaptide (100 nM; ▴▴▴P<0.001). Danegaptide partially reversed the TGFβ1 evoked change in Integrin Linked Kinase 1 (ILK1; ▴▴▴P<0.001) but had negligible effect on Matrix Metalloproteinase 3 (MMP3). All bars correspond mean±SEM, (n=3) and representative blots are shown with expression normalised by re-probing for α-tubulin as a loading control.

FIG. 6: Danegaptide prevents TGFβ1-evoked changes in the expression of adipokines, chemokines, growth factors and interleukins.

An inflammation antibody array was used to assess regulation of 31 candidate inflammatory proteins in cell lysates from hPTEC cells treated with TGFβ1±danegaptide. Results are representative of 3 separate experiments and presented as mean±SEM, n=3 with key significances indicated (*P<0.05, **P<0.01, ***P<0.001; one-way ANOVA and Tukey's post-test).

FIG. 7: Danegaptide prevents TGFβ1-evoked changes in the secretion of adipokines, chemokines, growth factors and interleukins.

An inflammation antibody array was used to assess regulation of 31 candidate inflammatory proteins in supernatant from hPTEC cells treated with TGFβ1±danegaptide. Results are representative of 3 separate experiments and presented as mean±SEM, n=3 with key significances indicated (*P<0.05, **P<0.01, ***P<0.001; one-way ANOVA and Tukey's post-test).

### DETAILED DESCRIPTION

### Terms and Definitions

To facilitate the understanding of the following description, a number of definitions are presented in the following paragraphs.

The term “treatment”, as used anywhere herein comprises any type of therapy, which aims at terminating, preventing, ameliorating and/or reducing the susceptibility to a kidney disease as described herein. The clinical conditions used in the present disclosure are well recognized by the skilled person and identified in the International Classification of Diseases, ICD-11 by World Health Organization, WHO.

As used herein, “a therapeutically effective amount” refers to an amount that is capable of reducing the clinical symptoms and signs of disease and partly or wholly repair or resolve the underlying pathology and/or normalising physiological responses in a subject with the condition or pathology. Reduction of symptoms or normalization of physiological responses can be determined using methods known in the art and can vary with a given condition or pathology.

### Glomerular Filtration Rate (GFR):

The best measure of kidney function is the glomerular filtration rate (GFR).

GFR can be estimated from a blood sample by using equations (eGFR) based on the plasma concentration of creatinine or cystatin C. These methods are well known in the art and it is common practise to determine the GFR of a subject with an accuracy sufficient to support determining the disease stage of a patient with chronic kidney disease, e.g. a human patient suffering from CKD.

### GJIC Modulator Compounds

The medical uses and methods of treatment of the present invention generally employ 1-(2-aminoacetyl)-4-benzoylamino-pyrrolidine-2-carboxylic acid, the compound of formula (I), also known as Danegaptide:

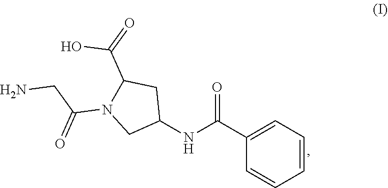 [SMILES: NCC(=O)N1CC(NC(=O)c2ccccc2)CC1C(=O)O]

or a pharmaceutically acceptable salt and/or hydrate thereof.

In particular, the medical uses and methods of treatment of the present invention employ the (2S,4R) diastereomer of this compound, as represented by the formula below:

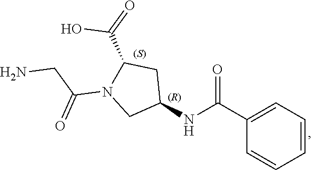

or a pharmaceutically acceptable salt and/or hydrate thereof.

An alternative name for this compound is (2S,4R)-1-(2-aminoacetyl)-4-10 benzamidopyrrolidine-2-carboxylic acid.

The preparation of 1-(2-aminoacetyl)-4-benzoylamino-pyrrolidine-2-carboxylic acid, such as the (2S,4R) diastereomer thereof, suitable methods of synthesis and purification thereof are described in WO 2007/078990, in which the (2S,4R) isomer is denoted “Compound 2” (WO 2007/078990 is incorporated by reference in its entirety).

An example of a useful salt form of the (2S,4R) diastereomer is the hydrochloride monohydrate, the preparation of which is described in WO 2008/079266, and which is also referred to in the present description as Compound X (WO 2008/079266 is incorporated by reference in its entirety).

The compounds for use in the present invention may contain two or more asymmetric atoms (also referred to as chiral centers), giving rise to the possibility of the occurrence of diastereomers. The medical uses and methods of the present invention include the use of these diastereomers.

Pharmaceutically acceptable salt of danegaptide include danegaptide hydrochloride, in particular danegaptide hydrochloride monohydrate.

### Pharmaceutically Acceptable Salts

Pharmaceutically acceptable salts of compounds suited for use in accordance with the invention having an acidic moiety can be formed using organic or inorganic bases. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di- or trihydroxy lower alkylamine (e.g., mono-, di- or triethanolamine). Internal salts also can be formed. When a compound suited for use in accordance with the invention contains a basic moiety (as in the case of, e.g., 1-(2-aminoacetyl)-4-benzoylaminopyrrolidine-2-carboxylic acid and the recited diastereomers thereof), salts may be formed using organic or inorganic acids. For example, salts can be formed from the following acids: acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic or camphorsulfonic. Other known pharmaceutically acceptable acids may also be employed. As already mentioned (vide supra), a preferred salt form of the (2S,4R) diastereomer of 1-(2-aminoacetyl)-4-benzoylamino-pyrrolidine-2-carboxylic acid is the hydrochloride monohydrate.

Pharmaceutically acceptable salts may also be formed from acids which form non-toxic addition salts containing pharmaceutically acceptable anions, such as the hydrochloride, hydrobromide, pamoate, hydroiodide, sulfate, or bisulfate, phosphate or acid phosphate, acetate, maleate, fumarate, oxalate, lactate, pamoate, tartrate, citrate, gluconate, saccharate and p-toluene sulphonate salts. In one embodiment the pharmaceutically acceptable salt of the compound according to the present disclosure is a hydrochloride salt.

The present teachings may also extend to the use of prodrugs of the compounds disclosed herein as being suited for use in accordance with the present invention. As used herein, “prodrug” refers to a moiety that produces, generates or releases a compound of one of the disclosed types when administered to a mammalian subject, notably a human subject. Prodrugs can be prepared by modifying functional groups present in the compounds in such a way that the modifications are cleaved, either by routine manipulation or in vivo, from the parent compounds. Examples of prodrugs include compounds as disclosed herein that contain one or more molecular moieties appended (bonded) to a hydroxy, amino, sulfhydryl or carboxy group of the compound, and that when administered to a subject to be treated are cleaved in vivo to form the free hydroxy, amino, sulfhydryl or carboxy group, respectively. Examples of prodrugs include, but are not limited to, acetate, formate and benzoate derivatives of alcohol and amine functional groups in the compounds disclosed herein for use in accordance with the invention. Examples of preferred prodrugs include oxazolidinone or imidazolidinone prodrugs. Ester prodrugs may be formed with lower alcohols, such as C06 alcohols. The preparation and use of prodrugs is discussed in T. Higuchi and V. Stella, “Pro-drugs as Novel Delivery Systems,” Vol. 14 of the A.C.S. Symposium Series, and in Bioreversibie Carriers in Drug Design, ed. Edward B. Roche, American Pharmaceutical Association and Pergamon Press, 1987.

### Pharmaceutical Compositions

Compounds, or pharmaceutically acceptable salts or hydrates thereof, employed in accordance with the present invention may be administered in the form of appropriate pharmaceutical compositions, which can be administered via any acceptable method known in the art, either singly or in combination.

Pharmaceutical compositions of relevance in the present context may comprise a compound as disclosed herein for use in accordance with the invention in admixture with one or more pharmaceutically acceptable carriers, diluents, vehicles or excipients.

The doses the compounds and compositions of the present invention required for the desired therapeutic effects will depend upon on the potency of the compound, the particular composition, used and the route of administration selected. The compounds will typically be administrated in the range of about 10 mg to about 500 mg per patient per day, such as from 10 mg to 100 mg. For example, the compounds can be administered in the range from about 50 mg to about 100 mg per patient per day, for example about 50 mg per patient per day. Alternatively, the compounds can be administered in the range from about 50 mg to about 150 mg per patient per day.

Administration of a compound (or pharmaceutical salt or hydrate thereof) in accordance with the invention may be conducted in a single unit dosage form (e.g. in the form of a bolus) or as a continuous therapy in the form of multiple doses over time. Alternatively, continuous infusion systems or slow release depot formulations may be employed. Two or more compounds for use in accordance with the invention (or pharmaceutical compositions thereof) may be co-administered simultaneously or sequentially in any order. In addition, the compounds and compositions may be administered in a similar manner for prophylactic purposes, for example if a diabetic patient deemed to be at risk of developing diabetic nephropathy or diabetic kidney disease. Ultimately, the best dosing regimen will be decided by the attending physician for each patient individually.

In accordance with the present disclosure, the compound may be administered by any suitable means to treat the kidney disease. In one embodiment, the compound is administered to the subject by oral administration. In one embodiment, the compound is administered by parenteral administration.

The compound in accordance with the invention may be administered daily, such as once daily (QD), twice daily (BID), three times daily (TID), or four times daily (QID). Preferably, the compound is administered once or twice daily.

### Therapeutic Uses

The medical uses and methods of treatment of the present invention are used for the treatment or prevention of kidney disease. Generally, the compounds are particularly useful where the kidney disease presents with renal inflammation and/or the development of renal fibrosis.

Alternatively or additionally, the present invention relates to the treatment or prevention of kidney disease that are types of chronic kidney disease (CKD), especially in a human subjects. In some cases the chronic kidney disease may present in a patient with renal fibrosis development and impaired kidney function. In some aspects, the chronic kidney disease is chronic kidney disease (CKD) especially in a human subject.

CKD can be subdivided into different disease stages according to ICD-11.
Stage 1: Kidney damage with normal or increased GFR (>90 ml/min/1.73 m²)
Stage 2: Kidney damage and GFR 60-89 ml/min/1.73 m²
Stage 3a: GFR 45-59 ml/min/1.73 m²
Stage 3b: GFR 30-44 ml/min/1.73 m²
Stage 4: GFR 15-29 ml/min/1.73 m²
Stage 5: Kidney failure, GFR<15 ml/min/1.73 m²
Stage Unspecified

In a preferred embodiment, the compound of the present disclosure provides an effective treatment of CKD stages 1 through 4. In one embodiment the compound of the present disclosure is used for treatment of CKD, wherein the CKD is at stage 1, defined by kidney damage with normal or increased glomerular filtration rate (GFR), i.e. a GFR>90 ml/min/1.73 m² of the subject. In another embodiment the CKD is at stage 2, defined by kidney damage and GFR 60-89 ml/min/1.73 m² of the subject. In a further embodiment the CKD is at stage 3, defined by GFR 45-59 ml/min/1.73 m² of the subject. In another embodiment the CKD is at stage 4, defined by GFR 15-29 ml/min/1.73 m² of the subject.

The compound is particularly effective prior to the occurrence of significant tissue damage as is seen in stage 5.

In some cases, the compound may be for use in the prevention of progression of CKD. In some embodiments, the compound prevents the progress from one Chronic Kidney Disease (such as at any one of stages 1, 2, 3a, 3b, or 4) to a more advanced Chronic Kidney Disease stage. In some embodiments, the compound prevents the progress from Chronic Kidney Disease at stage 1 to a Chronic Kidney Disease at any one of stages 2, 3a, 3b, 4, or 5. In some embodiments, the compound prevents the progress from Chronic Kidney Disease at stage 2 to a Chronic Kidney Disease at any one of stages 3a, 3b, 4, or 5. In some embodiments, the compound prevents the progress from Chronic Kidney Disease at stage 3a to a Chronic Kidney Disease at stage 3b, 4 or 5. In some embodiments, the compound prevents the progress from Chronic Kidney Disease at stage 3b to a Chronic Kidney Disease at stage 4 or 5. In some embodiments, the compound prevents the progress from Chronic Kidney Disease at stage 4 to a Chronic Kidney Disease at stage 5.

In another embodiment the compound for use according to the present invention is used in the treatment of diabetic kidney disease. This can also be termed diabetic nephropathy and represents kidney damage that results from having diabetes. In some patients diabetic nephropathy can result in CKD and kidney failure.

The compound of the present disclosure can furthermore be used for the treatment of a kidney disease, wherein the kidney disease is caused by an underlying disease selected from the group consisting of: Immune-mediated diseases, connective tissue diseases, Systemic lupus erythematosus (lupus nephritis), Diabetic Nephropathy, Sarcoidosis, Sjögren's syndrome, Amyloidosis, Multiple myeloma, Vasculitis, Cancer and Genetic disorders (such as congenital nephrotic syndrome), Atresia or Stenosis of the Ureter, Calculus (kidney stone) of Upper or Lower urinary tract, or Obstructive and Reflux Nephropathy.

Other relevant indications for which the compound of the present applications can be used are Renal Tubulo-Interstitial Disease. In some patients, Renal Tubulo-Interstitial Disease may lead to the development of Chronic Kidney Disease (CKD). Specific examples of such diagnoses of relevance are Chronic Tubulo-Interstitial Nephritis, Drug- and Heavy-Metal-Induced Tubulo-Interstitial and Tubular Conditions, Renal Tubulo-Interstitial Disorders in Systemic Connective Tissue Disorders and Renal Tubulo-Interstitial Disorders in transplant rejection.

The present disclosure further relates to a compound as defined herein for use in the treatment of a kidney disease, wherein the kidney disease is a rare inherited kidney disease, which presents with inflammation and fibrosis. In some patients, the rare kidney disease may lead to the development of Chronic Kidney Disease (CKD). Polycystic kidney disease (PKD) is a relevant example of a rare kidney disease, which presents with inflammation and development of fibrosis. PKD is a genetic disorder characterized by formations of numerous cysts in kidneys and most caused by PKD1 or PKD2 mutations in autosomal dominant polycystic kidney disease (ADPKD). The interstitial inflammation and fibrosis is one of the major pathological changes in polycystic kidney tissues with an accumulation of inflammatory cells, chemokines, and cytokines. The immune response is observed across different disease stages and occurs prior to or coincident with cyst formation in ADPKD. Evidence for inflammation as an important contributor to cyst growth and fibrosis includes increased interstitial macrophages, upregulated expressions of pro-inflammatory cytokines, activated complement system, and activated pathways including NF-κB and JAK-STAT signaling in polycystic kidney tissues. Inflammatory cells are involved in the overproduction of several pro-fibrotic growth factors which promote renal fibrosis in ADPKD. These growth factors trigger epithelial mesenchymal transition and myofibroblast/fibrocyte activation, which stimulate the expansion of extracellular matrix (ECM) including collagen I, III, IV, V, and fibronectin, leading to renal fibrosis and reduced renal function. There are imbalanced ECM turnover regulators which lead to the increased ECM production and inadequate degradation in polycystic kidney tissues. Although the effective anti-fibrotic treatments are limited at the present time, slowing the cyst expansion and fibrosis development may be very important for prolonging life span and improving the care of patients with ADPKD. The inhibition of pro-fibrotic cytokines involved in fibrosis might be a new therapeutic strategy for patients with ADPKD.

Temporary or chronic stenosis or obstruction of the ureter is another known condition that would lead to CKD and fibrosis development. These could be conditions such as Atresia or Stenosis of the Ureter, or Calculus (kidney stone) of Upper or Lower urinary tract, or Obstructive and Reflux Nephropathy.

Generally, it is known that 3 key factors are involved in the development of CKD; elevated blood pressure, high glucose levels in blood and inflammation. Thus, treatment with a compound as disclosed herein can be used in patients suffering from damage to the kidneys due to high blood pressure, such as Essential Hypertension or Hypertensive Renal Disease or damage to the kidneys due to hyperglycemia, such as diabetes associated Elevated Blood Glucose Levels.

An objective of treating a patient with a compound disclosed herein is to prevent further deterioration of kidney function, and development into End Stage Renal Disease (ESRD) and kidney failure. Once a patent develops kidney failure, treatment options are limited and include invasive procedures such as haemodialysis, peritoneal dialysis and kidney transplantation.

A patient recently diagnosed with diabetes mellitus may be a good candidate for treatment by a compound disclosed herein for several reasons. Some of the clinical manifestations often observed at the time of initial diagnosis of diabetes is proteinuria and elevated glucose levels in the urine, suggesting ongoing cellular stress on the kidney tissues. Patients with Type 1 diabetes typically experience a period called the ‘honeymoon phase’ where treatment with exogenous insulin is temporarily sufficient to offset the loss in secretory capacity and potentially reduce the ongoing loss of beta-cell mass. This seems to reduce the stress on the beta-cells allowing them to recover from an ongoing immune attack. Administering a compound disclosed herein to a patient in this period may further help to protect the remaining beta-cell mass in addition to protecting the kidneys from ongoing cellular stress, inflammation and fibrosis development. The data presented herein supports that a patient recently diagnosed with diabetes mellitus and with ongoing stress in the kidney tissues may benefit from a period of treatment with a compound disclosed herein reducing the inflammatory and profibrotic conditions in the kidneys.

In the kidney, reduced E-cadherin mediated cell adhesion initiates a series of events that culminate in an intermittent phenotype associated with partial epithelial-to-mesenchymal transformation. Initiation facilitates disassembly of both adherens and tight junction complexes, culminating in loss of adhesion, diminished gap junction intercellular communication and a leaky tubular epithelium. The use of a compound disclosed herein can restore changes in adherens and tight junction proteins and paracellular permeability. Furthermore, the use of a compound according to the present disclosure can inhibit hemi-channel ATP release, such as Cx43 mediated ATP release.

Caused by an imbalance between formation and degradation, the accumulation of extracellular matrix (ECM) is a major hallmark of CKD. A role for TGFβ1 in this process is well established (for example, see Meng et al., Nat Rev Nephrol, 2016, 12, 325-338; and Akagi, Y et al., Kidney International, 1996, Vol. 50: 148-55) and understanding how to negate these changes has clear implications for how to avoid or reduce progression of the disease.

The inflammatory response in and around proximal tubules involves both activation of multiple cell types combined with the secretion of numerous inflammatory markers. Specifically, soluble chemokines, cytokines and growth factors recruit and activate infiltrating immune cells and resident fibroblasts. Sustained activation of these cells mediates tubulointerstitial fibrosis.

Accordingly, in one aspect, the compound of the present disclosure restores changes in expression and secretion of extracellular matrix proteins, adipokines, chemokines and growth factors induced by TGFβ1. In another aspect, the compound of the present disclosure decreases inflammation in the proximal tubule or tubulointestinal tissues. In a further aspect, the compound of the present disclosure protects against changes in renal function associated with inflammation and fibrosis.

Cellular senescence may play a key role in progression of Chronic Kidney Disease with senescence being linked to EMT, a pro-inflammatory secretome and extracellular matrix deposition. Senescence denotes irreversible proliferative growth arrest with associated changes in chromatin organization, gene transcription, and protein secretion. In this context, senescent cells are known to exhibit increased expression of cyclin-dependent kinase (CDK) inhibitors (CKIs) including p21Cip1 (p21), and p161nk4a (p16), and altered expression of reno-protective Klotho. With adoption of a senescent phenotype preceding development of functional changes associated with kidney damage, clearance of senescent cells in kidney disease may therefore improve renal function. Data presented herein i.a. in Example 4 shows that TGFβ1 induces increased expression of p16, p21 and cyclin D1 and that these changes in expression were restored when cells were co-incubated with danegaptide. These observations support that danegaptide, or a pharmaceutically acceptable salt thereof, is useful in the treatment of a kidney disease, such as Chronic Kidney Disease (CKD).

According to the present invention, danegaptide, or a pharmaceutically acceptable salt thereof (e.g. in the form of an appropriate pharmaceutical composition), may be administered to a subject in need thereof in a therapeutically effective amount.

The effective amount of the compound can be at least about 0.1 mg/kg body weight/day, such as at least about 0.3 mg/kg body weight/day, at least about 0.5 mg/body weight/day, such as at least about 1 mg/kg body weight/day, such as at least about 2 mg/kg body weight/day. On the other hand, the effective amount of the compound or dimer can be at most about 10 mg/kg body weight/day, such as at most about 5 mg/kg body weight/day and at most about 3 mg/kg body weight/day. It is expected that the effective amount of the compound will be about 0.5 mg/kg body weight/day, about 1 mg/kg body weight/day, about 2 mg/kg body weight/day, or about 5 mg/kg body weight.

The experiments provided in the in vitro systems described herein, dose finding as performed in the in vitro systems (carboxyfluorescein dye uptake in HK2 cells and ATP biosensing in HK2 cells), show that a concentration of 50-100 nM is optimal in the microenvironment and danegaptide was confirmed not to have negative effects on cell viability in the assays. Thus, in one embodiment the use of the compound of the present disclosure provides for a concentration of the administered compound of about 50 nM to about 100 nM in the microenvironment of the renal tissues.

The subject(s)/patient(s) treated according to the present invention is preferably human and can be of any age, i.e. an infant, a child, an adolescent, an adult, or an elderly.

### Method of Selecting a Subject for Treatment

In one embodiment, a method for selecting a subject for treatment by a compound disclosed herein is provided, said method comprising providing a sample comprising a kidney disease biomarker. In some embodiments, this method further comprises treating the subject with the compound. Any means known in the art that allows diagnosis of the clinical diseases described herein, such as a chronic kidney disease, are suitable to select a subject for treatment by a compound of the present disclosure.

In one embodiment, a method for selecting a subject for treatment by the compound disclosed herein is provided, said method comprising:
a. Providing a sample or a biopsy from the subject,
b. Determining thickening of basal membrane (BM) and/or
c. Mesangial matrix expansion, relative to a baseline level from a control sample,
   wherein a thickened basal membrane and/or an expanded mesangial matrix indicates that the subject is responsive to treatment with the compound.

In one embodiment the sample from the subject is selected from the group consisting of: a sample comprising diseased tissue or diseased cells from the kidney, blood sample, urine sample, biopsy sample and tissue resection. In a specific embodiment the sample from the subject is a sample comprising diseased tissue or diseased cells from the kidney. In another embodiment the sample is a blood sample. In a further embodiment the sample is a urine sample. In one embodiment, the control sample is obtained from one or more healthy subjects and comprises healthy tissue or healthy cells of the same origin as the disease tissue or diseased cells.

Another simple and relevant diagnostic marker for selection of a patient for treatment by a compound disclosed herein is the measurement of protein or albumin in a urine sample from a patient. Persistent Proteinuria or Albuminuria would be a clinical indication in which danegaptide would have therapeutic potential. Sometimes early manifestations are described as a patient having micro-albuminuria. Measurement of albuminuria, urine albumin-to-creatinine ratio or micro-albuminuria are particular useful diagnostic markers supporting the selection of a patient for treatment by a compound disclosed herein. The measurements of albuminuria and micro-albuminuria may appropriately be supplemented with other diagnostic markers such as presence of hypertension, dyslipidemia and haemoglobin A1c levels to further optimise selection of a patient for treatment by a compound disclosed herein. Measurement or detection of inflammatory cells or red blood cells in the urine are known markers of nephritis and may be relevant supportive markers to select a patient for treatment. Presence of other inflammatory kidney disease biomarkers such as monocyte chemoattractant protein-1 (MCP-1), neutrophil gelatinase-associated lipocalin (NGAL), kidney injury molecule-1 (KIM-1), Regulated on Activation Normal T-cells Expressed and Secreted chemokines (RANTES), tumour necrosis factor alpha (TNF-α), matrix metallopeptidase 9 (MMP9), intercellular adhesion molecule 1 (ICAM 1), klotho, asymmetric dimethylarginine (ADMA), and certain cytokines such as interleukin-18 (IL-18), transforming growth factor beta 1 (TGFβ1), granulocyte-colony stimulating factor (G-CSF), granulocyte-macrophage colony-stimulating factor (GM-CSF), insulin-like growth factor binding protein 2 (IGFBP2), insulin-like growth factor binding protein 3 (IGFBP3), and leukemia inhibitory factor (LIF) may also be useful in predicting whether the subject is responsive to treatment with the compounds disclosed herein. For further examples of biomarkers in chronic kidney disease (CKD), see Fassett et al., Kidney International, 2011, 80, 806-821; Lopez-Giacoman et al., World J Nephrol, 2015, 4(1), 57-73; Lousa et al., Int. J. Mol. Sci., 2021, 22, 43. Example 7 described herein shows that the compound of the present disclosure modulates these kidney disease biomarkers and other inflammatory markers in primary human proximal tubular epithelial cells (primary hPTECs) exposed to TGFβ1-induced stress, demonstrating a protective effect against kidney disease, such as chronic kidney disease (CKD).

In one embodiment the compound of the present disclosure is used in the treatment of a kidney disease, wherein the kidney disease presents with proteinuria, albuminuria or micro-albuminuria.

On a biopsy from the kidney tissue from a patient with a kidney disease, one may observe damage to the kidneys due to glomerulonephritis, interstitial nephritis or polycystic kidney disease and these observations may also serve to guide the selection of a patient for treatment by a compound disclosed herein. In one embodiment the compound of the present disclosure is used in the treatment of a kidney disease, wherein the kidney disease is a glomerular disease. In a further embodiment the glomerular disease is glomerulosclerosis or diabetic glomerulosclerosis.

Combinations of these diagnostic markers and the inclusion of markers of inflammation may further help to identify suitable subjects for treatment.

A patient suitable for treatment wherein the kidney disease presents with renal inflammation and/or development of renal fibrosis may be identified by different diagnostic markers and/or having certain symptoms. Patients may present with symptoms such as fever, chills, pain in the flank, abdomen and/or groin. Frequent urination or burning sensation or pain when urinating may also be symptoms suggesting that a patient may have renal inflammation. Diagnostic markers such as detection of red blood cells or inflammatory cells in a urine sample may support findings of renal inflammation. Use of a microscope, a urine dipstick or specific diagnostic tests, including biomarkers measured in urine or blood may reveal signs of inflammation and/or development of fibrosis.

Radiological methods, including ultrasonography and computer tomography (CT) may further support the findings of inflammation. Signs of kidney fibrosis may be diagnosed based on a kidney biopsy and relevant radiological methods include ultrasonography and magnetic resonance imaging (MRI).

In clinical practice, the identification of a patient with a kidney disease presenting with renal inflammation and/or renal fibrosis will often be based on a combination of a patient presenting with relevant symptoms and supported by several of such diagnostic markers.

### Items:

1. A compound of formula (I),

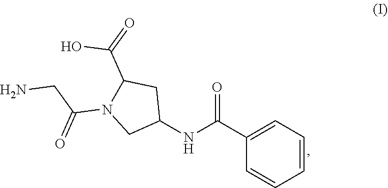 [SMILES: NCC(=O)N1CC(NC(=O)c2ccccc2)CC1C(=O)O]

or a pharmaceutically acceptable salt and/or hydrate thereof; for use in the treatment or prevention of a kidney disease.
2. The compound for use in the treatment or prevention according to item 1, wherein the kidney disease presents in a subject with renal inflammation and/or development of renal fibrosis.
3. The compound for use in the treatment or prevention according to any one of the preceding items, wherein the kidney disease is Chronic Kidney Disease (CKD) or Diabetic Nephropathy.
4. The compound for use in the treatment or prevention according to item 3, wherein the kidney disease is Diabetic Nephropathy that has resulted in Chronic Kidney Disease (CKD).
5. The compound for use in the treatment or prevention according to item 3 or item 4, wherein:
   (a) the Chronic Kidney Disease is at stage 1, defined by kidney damage and GFR>90 ml/min/1.73 m² of the subject; or
   (b) the Chronic Kidney Disease is at stage 2, defined by kidney damage and GFR 60-89 ml/min/1.73 m² of the subject; or
   (c) the Chronic Kidney Disease is at stage 3a, defined by GFR 45-59 ml/min/1.73 m² of the subject; or
   (d) the Chronic Kidney Disease is at stage 3b, defined by GFR 30-44 ml/min/1.73 m² of the subject; or
   (e) the Chronic Kidney Disease is at stage 4, defined by GFR 15-29 ml/min/1.73 m² of the subject.
6. The compound for use in the treatment or prevention according to any of the preceding items, wherein the kidney disease presents with proteinuria, albuminuria or micro-albuminuria.
7. The compound for use in the treatment or prevention according to any of the preceding items, wherein the kidney disease is a glomerular disease, such as glomerulosclerosis or diabetic glomerulosclerosis.
8. The compound for use in the treatment or prevention according to any one of the preceding items, wherein the kidney disease is caused by an underlying disease selected from the group consisting of: Systemic lupus erythematosus (lupus nephritis), Diabetic nephropathy, Sarcoidosis, Sjögren's syndrome, Amyloidosis, Multiple myeloma, Vasculitis, Atresia or Stenosis of the Ureter, Calculus (kidney stone) of Upper or Lower urinary tract, or Obstructive and Reflux Nephropathy.
9. The compound for use in the treatment or prevention according to any one of the preceding items, wherein the kidney disease is Renal Tubulo-Interstitial Disease, such as Chronic Tubulo-Interstitial Nephritis, Drug- and Heavy-Metal-Induced Tubulo-Interstitial and Tubular Conditions and Renal Tubulo-Interstitial Disorders in Systemic Connective Tissue Disorders.
10. The compound for use in the treatment or prevention according to any one of the preceding items, wherein the kidney disease is a rare inherited kidney disease, such as polycystic kidney disease (PKD).
11. The compound for use in the treatment or prevention according to any one of the preceding items, wherein the compound inhibits hemi-channel ATP release, such as Cx43 mediated ATP release.
12. The compound for use in the treatment or prevention according to any one of the preceding items, wherein:
   (a) the compound restores changes in expression and secretion of extracellular matrix proteins, adipokines, chemokines and growth factors induced by TGFβ1; and/or
   (b) the compound protects against changes in renal function associated with inflammation and fibrosis; and/or
   (c) the compound decreases inflammation in the proximal tubule or tubulointestinal tissues.
13. The compound for use in the treatment or prevention according to any one of the preceding items, wherein the compound is represented by the formula:

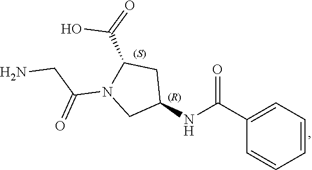

or a pharmaceutically acceptable salt thereof.
14. The compound for use in the treatment or prevention according to any one of the preceding items, wherein the use provides for a target concentration of the administered compound of 50-100 nM in the microenvironment of the renal tissues.
15. The compound for use in the treatment or prevention according to any one of the preceding items, wherein the pharmaceutically acceptable salt is a hydrochloride salt.
16. The compound for use in the treatment or prevention according to any one of the preceding items, wherein the compound is administered orally or parenterally, preferably wherein the compound is administered orally.

### EXAMPLES

### Materials

Clonal human kidney (HK2) epithelial cells and primary human proximal tubule epithelial cells (hPTECs) were purchased from ATCC (LGC Standards). Tissue culture supplies were purchased from Invitrogen (Paisley, UK). Immobilon-FI PVDF membrane was from Millipore (Watford, UK), and Odyssey blocking buffer and secondary fluorescent antibodies were purchased from LI-COR (Cambridge, UK). Antibodies for E-cadherin, N-cadherin and ZO-1 were obtained from Cell Signalling Technologies (Hertfordshire, UK), whilst Claudin-2, Col I and Col IV antibodies were obtained from ABCAM (Cambridge, UK). Fibronectin antibody was purchased from Santa Cruz (Santa Cruz, CA, USA). Recombinant hTGFβ1 was obtained from Sigma (Poole, UK), as were all other general chemicals. Danegaptide was provided by Zealand Pharmaceuticals. ATP biosensors were from Sarissa Biomedical Ltd (Coventry, UK) and fluorodishes from WPI (Hertfordshire, UK). Transwell filters were purchased from Corning (Nottinghamshire, UK). The Proteome Profiler Human Cytokine Array Kit was from R&D Systems (Oxfordshire, UK).

Primary human proximal tubule epithelial cells (hPTECs) were maintained in a renal epithelial cell basal medium from ATCC, supplemented with 0.5% FCS wt/vol, triiodothyronine (10 nM), rhEGF (10 ng/ml), hydrocortisone hemisuccinate (100 ng/ml), rhInsulin (5 μg/ml), epinephrine (1 μM), transferrin (5 μg/ml) and L-Alanyl-L-Glutamine (2.4 mM), in a humidified atmosphere at 37° C. with 5% CO₂. Cells were subjected to overnight serum-starvation prior to treatment. Human kidney (HK2) cells (passage 18-30) were grown in DMEM/Hams F12 medium, supplemented with 10% FCS wt/vol, glutamine (2 mmol/1) and EGF (5 ng/ml), in a humidified atmosphere at 37° C. with 5% CO₂. HK2 cells were immortalised by the transduction of human papilloma virus 16 (HPV-16) E6/E7 genes and are mycoplasma-free. In all experiments, cells were seeded in low glucose DMEM/F12 (5 mmol/L) for 48 h, then serum starved overnight prior to treatment.

### Example 1—Danegaptide does not Affect Tubular Epithelial Cell Viability

### Materials and Methods

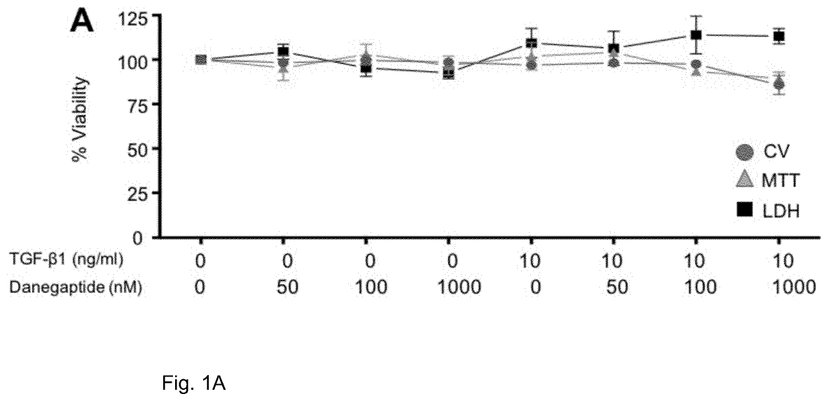

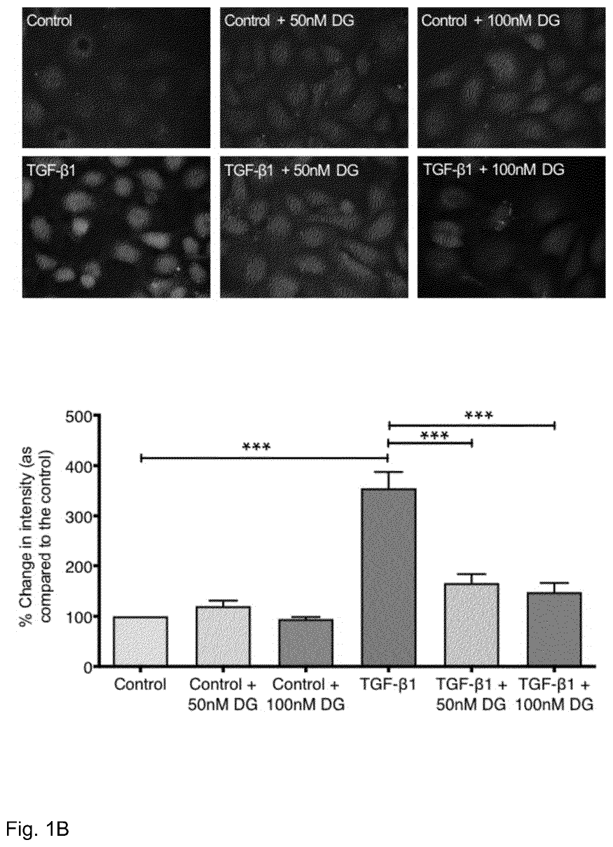

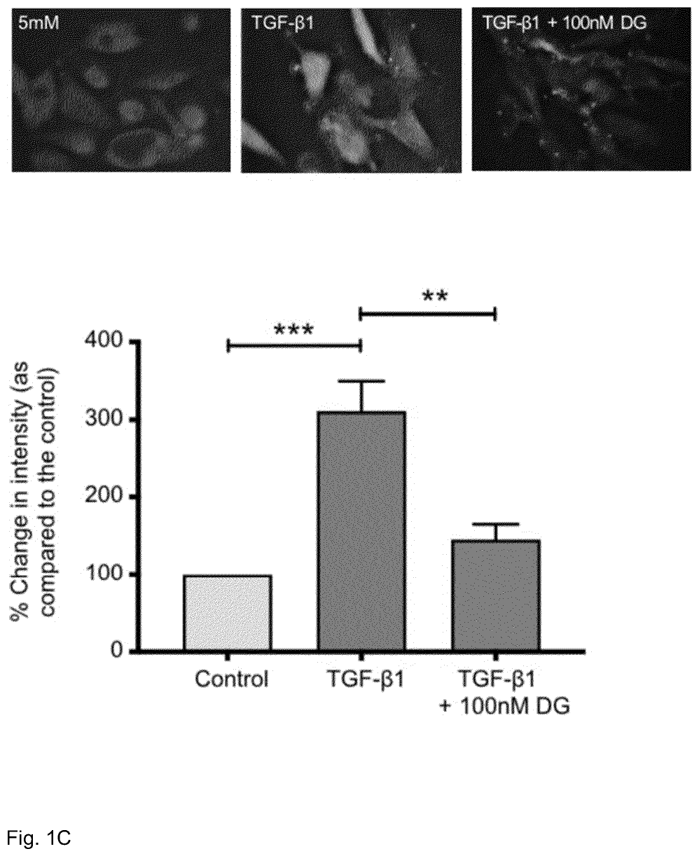

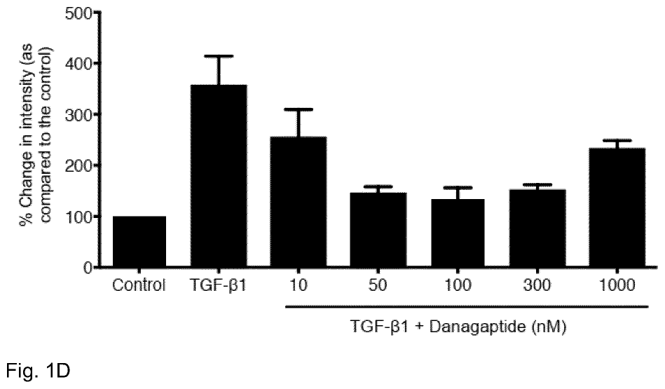

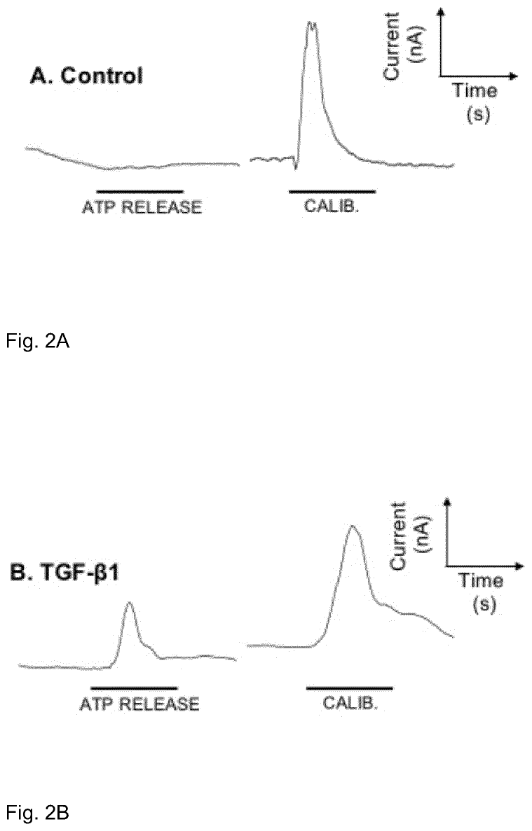

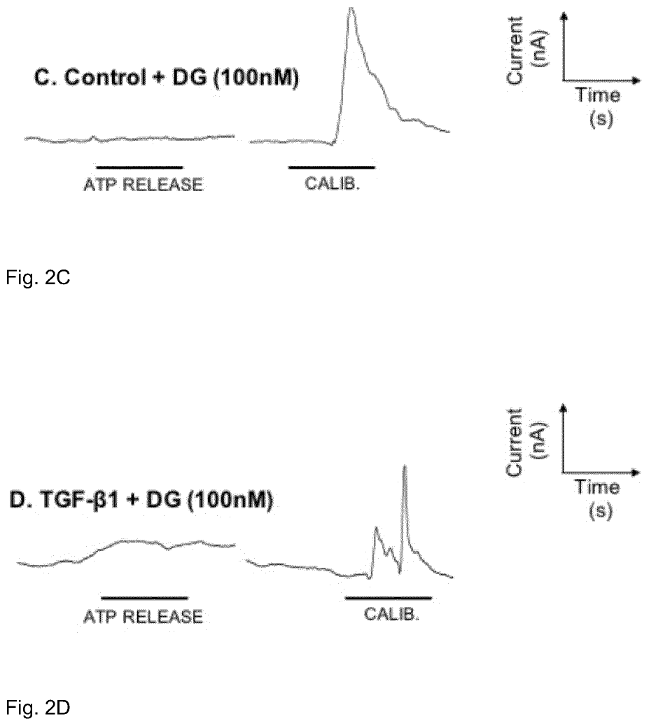

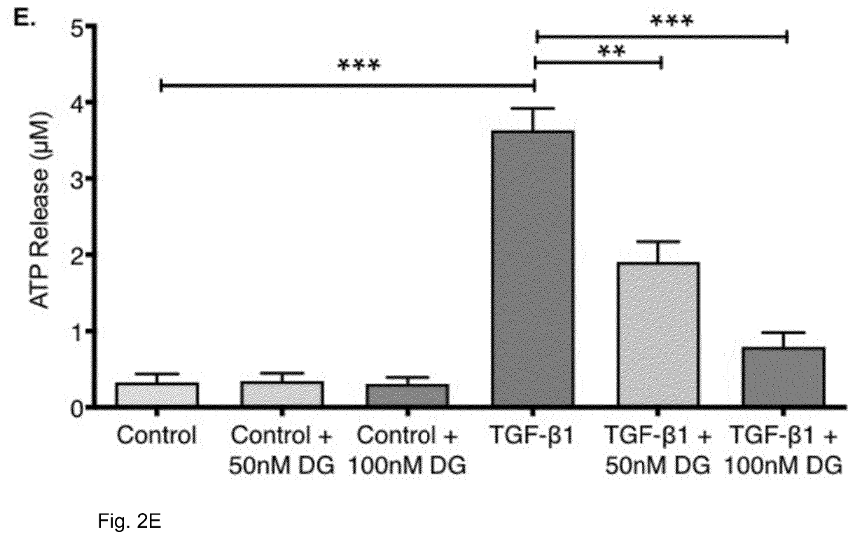

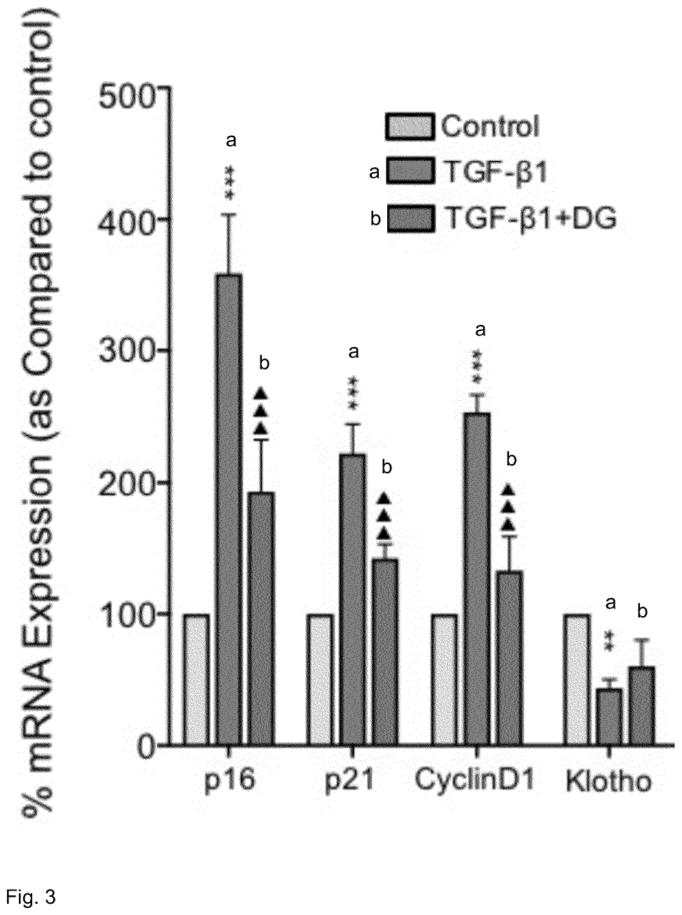

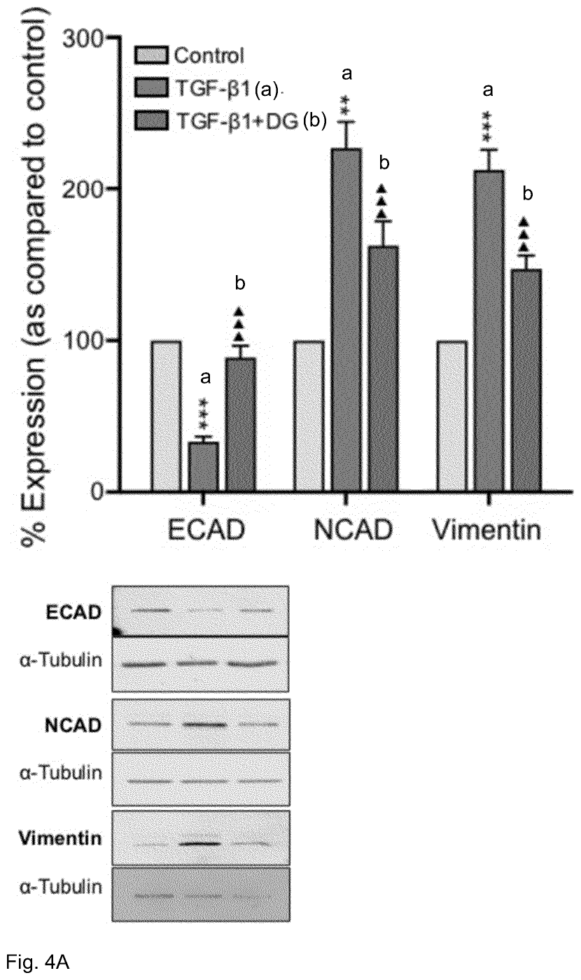

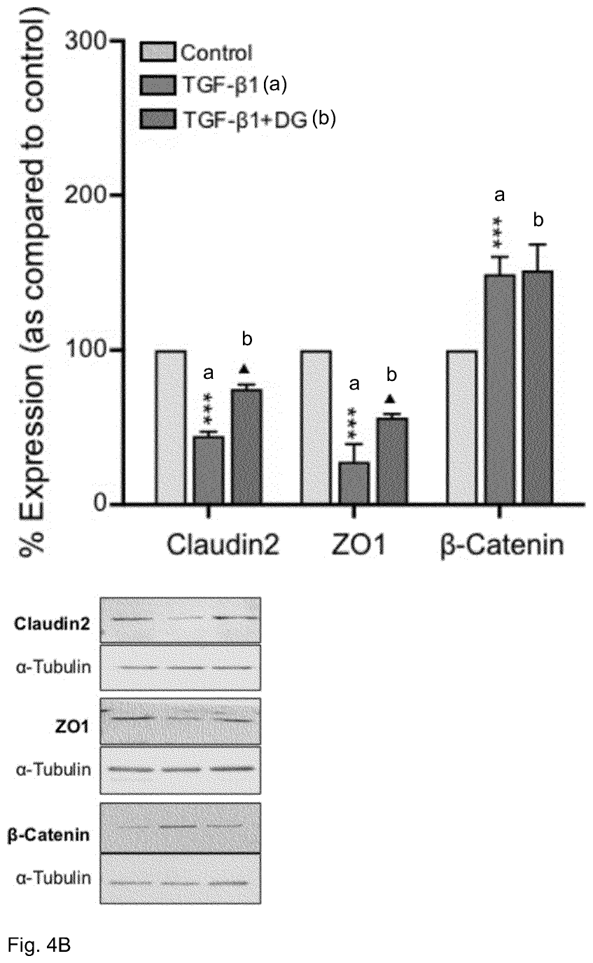

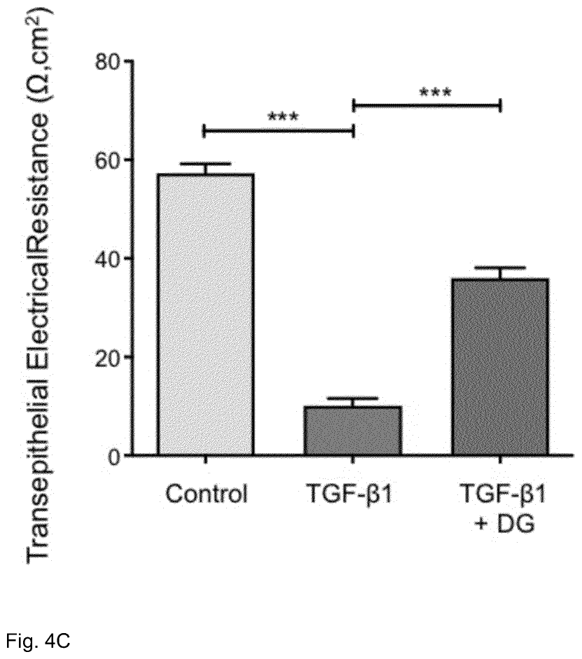

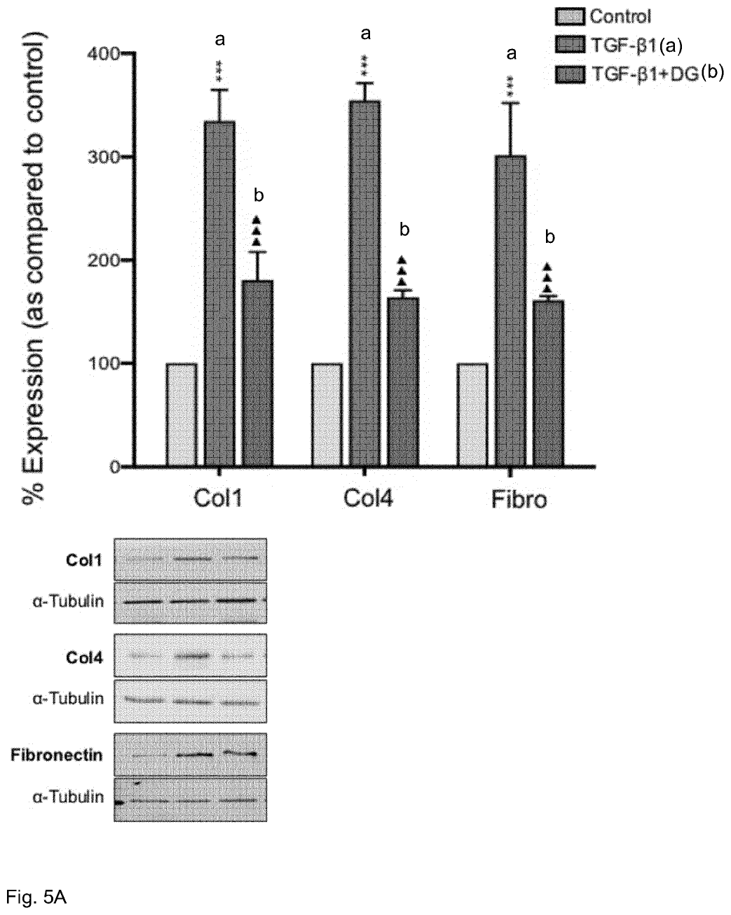

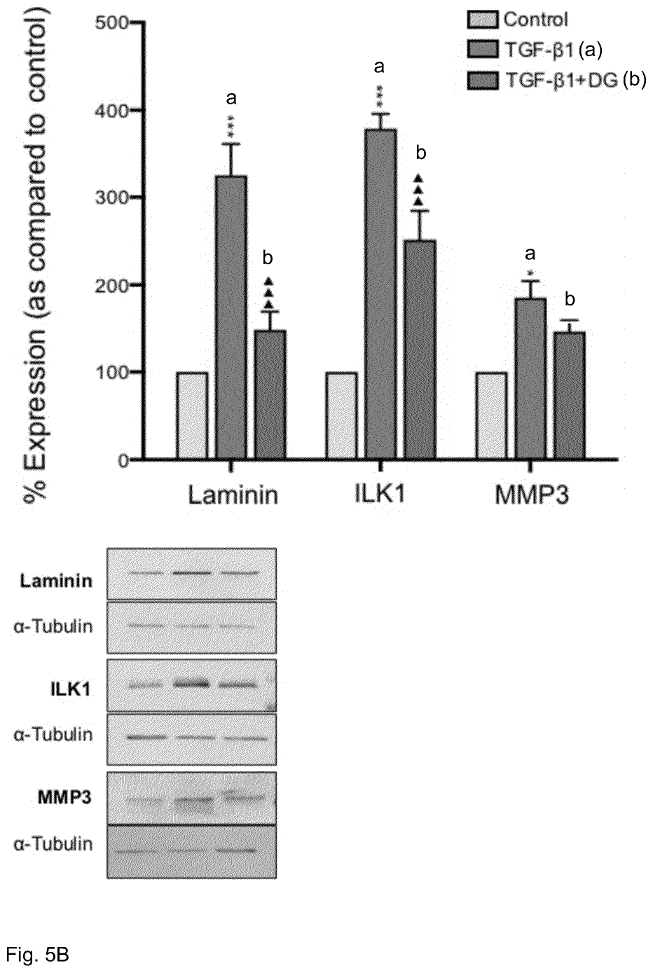

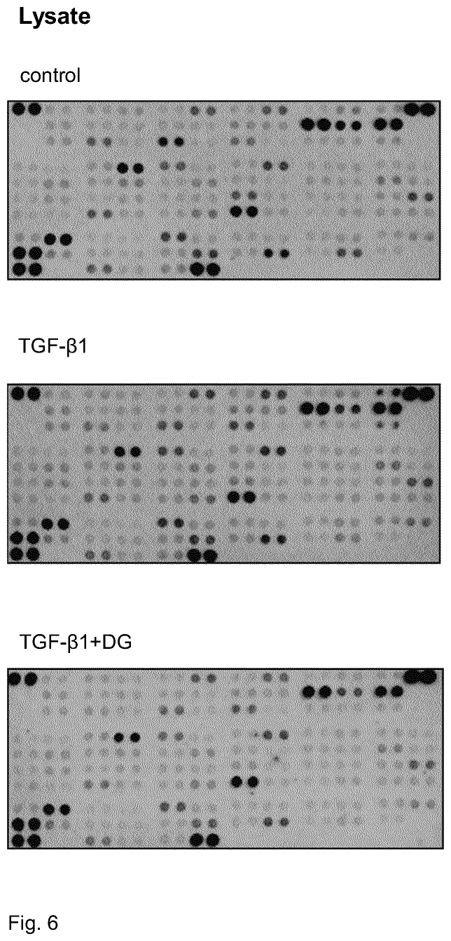

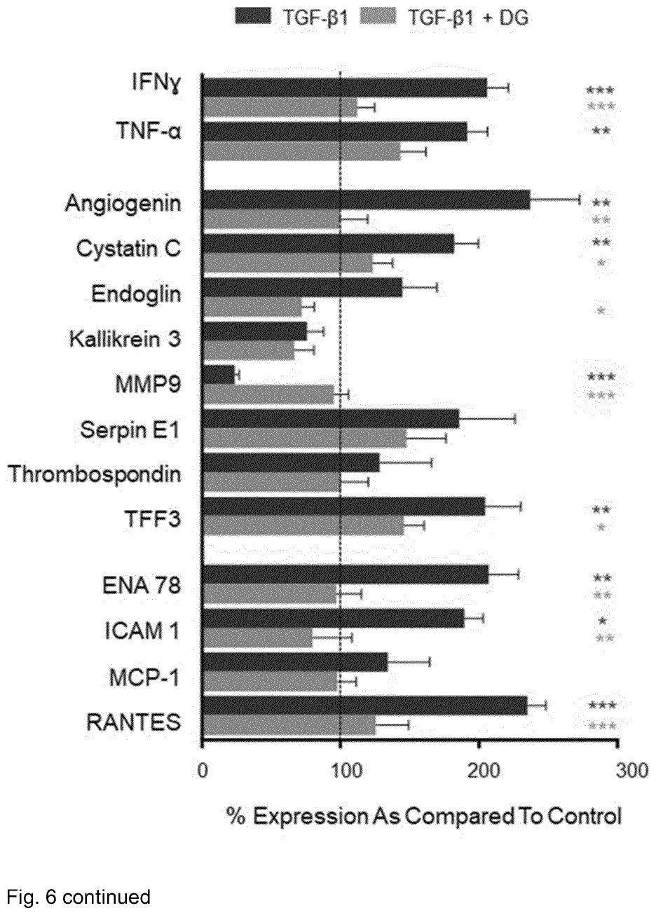

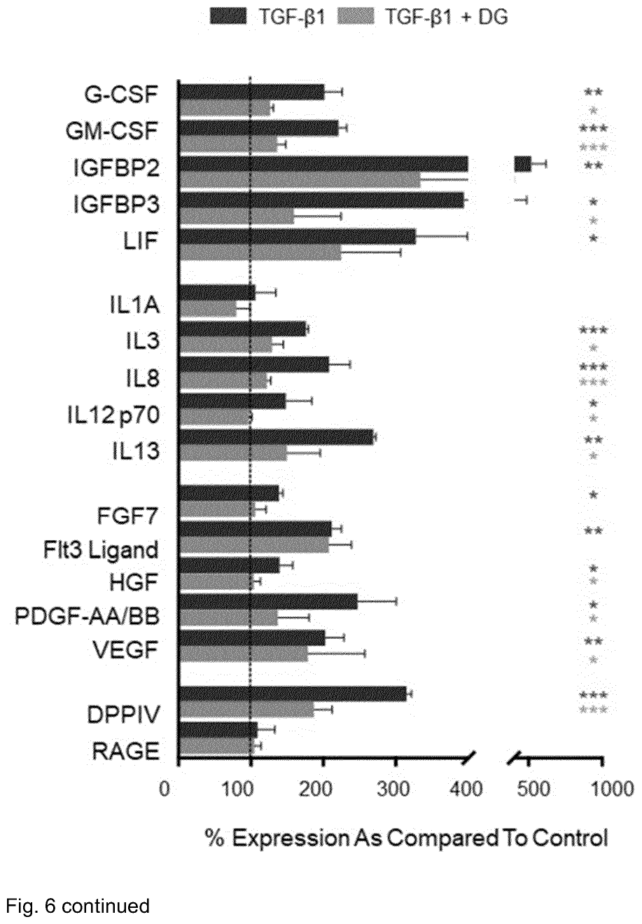

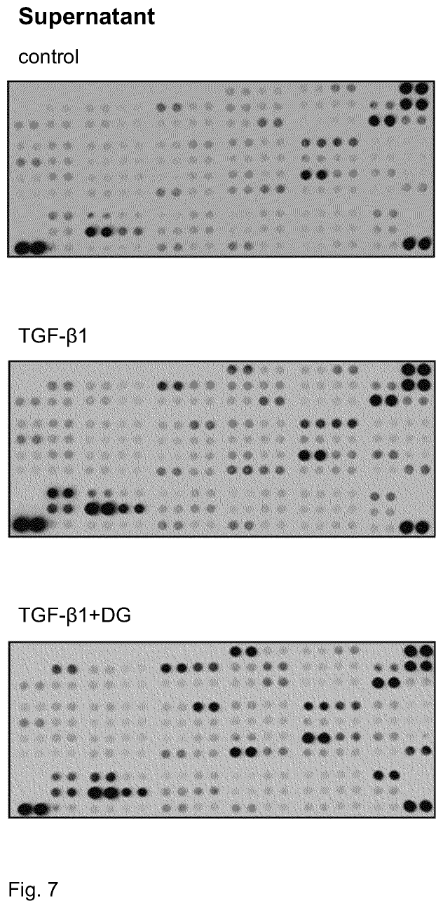

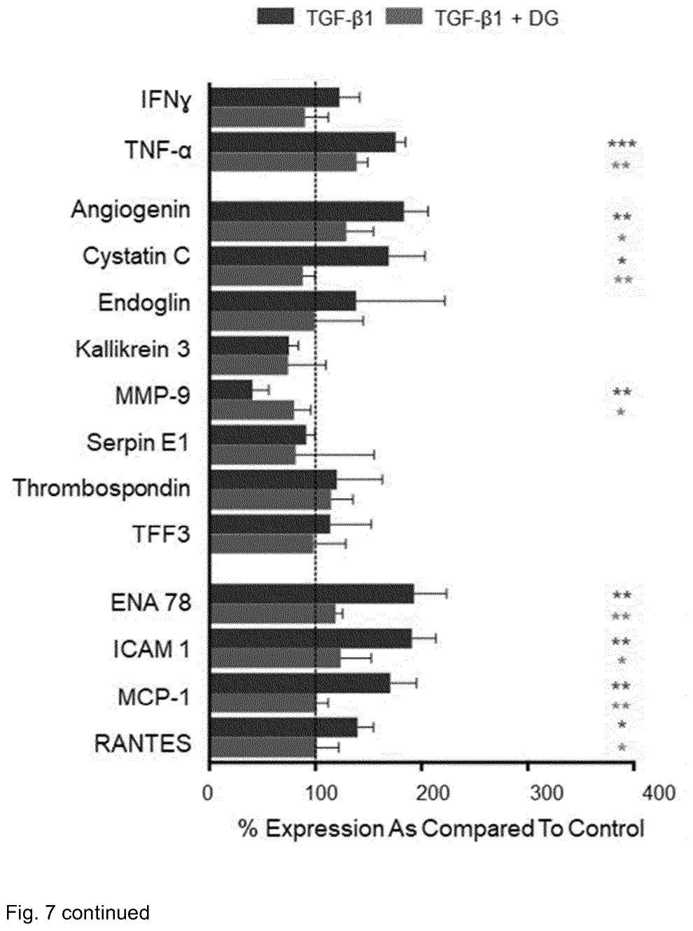

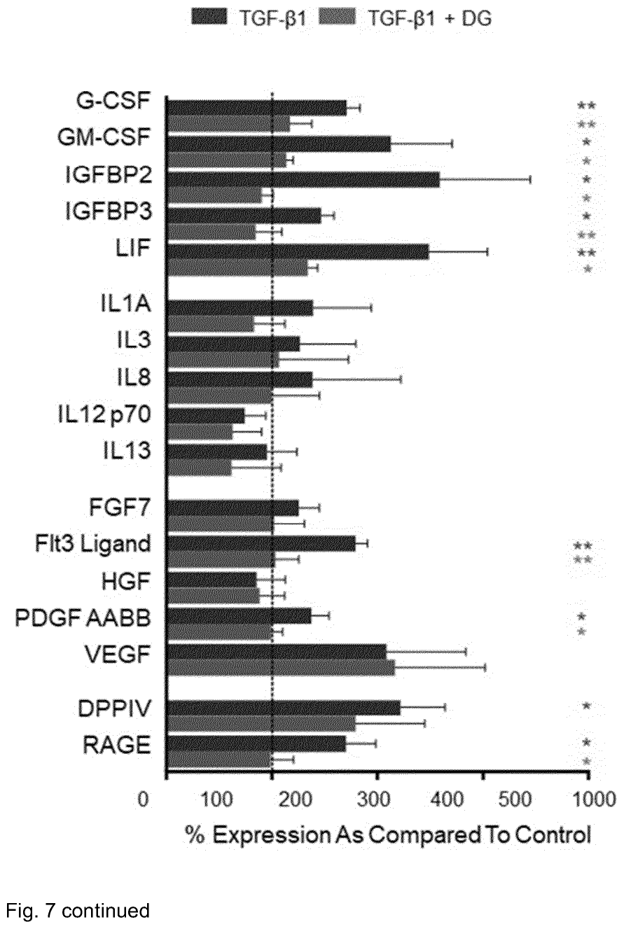

HK2 cells were cultured in low glucose (5 mM) for 48 h, prior to being serum-starved overnight and subsequently treated with TGFβ1 (10 ng/mL)±danegaptide (50 nM-1 μM) for 48 h (FIG. 1A, n=3).

### MTT Assay

The 3-(4,5-Dimethylthiazol-2-yl)-2,5-diphenyltetrazolium bromide (MTT) assay was performed as known in the art to assess the cytotoxic effects of danegaptide on cell proliferation. HK2 cells were seeded in 96-well plates and cultured in low glucose DMEM/F12 (5 mM) for 48 h, prior to overnight serum-starvation, then subsequently stimulated for 48 h with TGFβ1 (10 ng/ml)±danegaptide (50-1000 nM). Colourmetric measurement of formazan production corresponds to the number of viable cells.

### Lactate Dehydrogenase Assay

The release of lactate dehydrogenase (LDH) into media as a result of plasma membrane damage is commonly used to evaluate cell death or cytotoxicity. HK2 cells were seeded in 96-well plates and cultured in low glucose DMEM/F12 (5 mM) for 48 h, prior to overnight serum-starvation. Cells were then stimulated for 48 h with TGFβ1 (10 ng/ml)±danegaptide (50-1000 nM). The LDH-cytotoxicity assay kit II (Abcam) was used to quantify LDH according to the manufacturer's instructions.

### Crystal Violet Assay

This simple assay was used to measure the relative density of adhered cells to multi-well dishes. Crystal Violet stains DNA and can be quantified colourmetrically after solubilisation. HK2 cells were seeded in 12-well plates and cultured in low glucose DMEM/F12 (5 mM) for 48 h, prior to overnight serum-starvation, then subsequently stimulated for 48 h with TGFβ1 (10 ng/ml)±danegaptide (50-1000 nM). The assay has been described previously (Hills, C. E.; Jin, T.; Siamantouras, E.; Liulssac, I. K. K.; Jefferson, K. P.; Squires, P. E. “Special K” and a Loss of Cell-To-Cell Adhesion in Proximal Tubule-Derived Epithelial Cells: Modulation of the Adherens Junction Complex by Ketamine. PLoS One 2013, 8, e71819). Briefly, cells were fixed using paraformaldehyde for 10 mins, washed with PBS and incubated for 10 mins at RT in a 1% crystal violet solution. After several more washes, the stain was solubilised using 1% SDS, and absorbance measured by a plate reader.

### Results

The MTT assay confirmed that neither TGFβ1 (101.9±11.7%) nor danegaptide alone altered cell viability (95.2±7% (50 nM), 103±5.7% (100 nM) and 96.6±5.3% (1 μM)) as compared to control. No effect on cell viability was also observed when TGFβ1-treated cells were co-incubated with danegaptide (104.1±2.2% (50 nM), 93.4±1.6% (100 nM) and 89.3±3.7% (1 μM)). To corroborate these data, a crystal violet and LDH assay were performed. LDH release in TGFβ1 treated cells was comparable to control (109.3±11.3%) and co-incubation with danegaptide had no additional effect (106.4±11.6% (50 nM), 113.9±15.6% (100 nM) and 113.2±4.3% (1 μM)). As expected, danegaptide alone did not significantly alter LDH release compared to control (104.4±4.3% (50 nM), 95.3±4.7% (100 nM) and 92.6±3.3% (1 μM)). Cell staining using crystal violet recapitulated these findings, with data for TGFβ1 (10 ng/mL; 96.9±0.7.3%) and TGFβ1 plus danegaptide (50 nM-1 μM) being comparable to control (98.2±1.9% (50 nM), 97.5±1.8% (100 nM) and 85.8±5.3% (1 μM). Lastly, danegaptide alone, did not alter crystal violet staining (98.3±2.5% (50 nM), 99.6±2.7% (100 nM) and 98.5±2.2% (1 μM) of control). In light of these data, a concentration of 50-100 nM was selected for subsequent studies.

### Conclusion

This example demonstrates that cell viability was not adversely affected by the pro-fibrotic cytokine TGFβ1 used to stimulate the cells or by danegaptide administered as a gap junction modifier and hemi-channel blocker to modify TGFβ1-induced cellular responses.

### Example 2—Danegaptide Blocks TGFβ1-Evoked Changes in Hemichannel-Mediated Dye Uptake in Tubular Epithelial Cells

### Materials and Methods

Carboxyfluorescein Dye Uptake Assays

HK2 and HPTEC cells were seeded onto fluorodishes (22 mm diameter) and cultured in low glucose DMEM/F12 (5 mmol/1) for 48 h. Following an overnight serum-starvation, cells were incubated with TGFβ1 (10 ng/ml)±danegaptide (100 nM) for 48 h. For subsequent steps, a balanced salt solution (BSS, pH7.0) was used, comprising of NaCl (137 mM), KCl (5.4 mM), MgSO₄ (0.8 mM), Na₂HPO₄ (0.3 mM), KH₂PO₄ (0.4 mM), NaHCO₃ (4.2 mM), HEPES (10 mM) and glucose (5 mM). To induce dye uptake, cells were exposed to Ca²⁺-free BSS (zero CaCl₂+EGTA (1 mM)) plus carboxyfluorescein (200 μM) for 10 min, followed by a 10 min period in Ca²⁺-containing BSS (1.3 mM) plus carboxyfluorescein (200 μM). Dishes were subsequently washed with Ca²⁺-containing BSS (12 mls). Images were acquired with a Cool Snap HQ CCD camera (Roper Scientific) and Metamorph software (Universal Imaging Corp., Marlow, Bucks, UK). ImageJ was used to quantify dye uptake, where a region of interest (ROI) was drawn around each cell (approx. 10-15 cells/dish) and mean pixel intensity measured. A background fluorescence value was subtracted from each ROI.

### Results

The carboxyfluorescein dye uptake assay was used to determine if danegaptide can negate TGFβ1 induced dye uptake through hemichannels in HK2 cells and primary hPTECs. As expected, TGFβ1 (10 ng/mL) increased dye uptake to 354.9±32.6% of control in HK2 cells, whilst co-incubation with danegaptide (50 nM and 100 nM), significantly blunted the response 165.5±17.9% and 147.6±18.1% respectively (FIG. 1B; P≤0.001, n=4). Danegaptide alone did not alter dye uptake. Carboxyfluorescein uptake was increased by TGFβ1 (10 ng/mL) in primary hPTECs (310.8±38.6% of control), a response partly negated by the co-incubation of danegaptide (100 nM; 145±19.7%) as compared to control (FIG. 1C; P≤0.01, n=4). Finally, a dose range of 10 to 1000 nM danegaptide was tested and it was found that optimal inhibition of carboxyfluorescein dye uptake in HK2 Cells occurred at a concentration of 50-100 nM danegaptide (FIG. 1D).

### Conclusion

This example demonstrates that TGFβ1 (10 ng/mL) stimulates and stresses the renal epithelial cells to open their Cx43 based hemichannels allowing influx of Carboxyflurescein dye into the cells. Co-administration of danegaptide (100 nM) modified the Cx43 hemichannels to remain in a closed state and thereby limited the dye uptake as a marker of cellular stress. Hence, danegaptide or a pharmaceutically acceptable salt thereof, emerges as a promising candidate for protecting renal epithelial cells from TGFβ1-induced stress and pathological hemichannel opening.

### Example 3—Danegaptide Negates TGFβ 1-Induced Hemichannel Mediated ATP Release in hPTECs

### Materials and Methods

ATP Biosensing

ATP biosensors (Sarissa Biomedical, Coventry UK) were used in a simultaneous dual-recording ampomeric setup as described previously (Price, G. W.; Chadjichristos, C. E.; Kavvadas, P.; Tang, S. C. W.; Yiu, W. H.; Green, C. R.; Potter, J. A.; Siamantouras, E.; Squires, P. E.; Hills, C. E. Blocking Connexin-43 mediated hemichannel activity protects against early tubular injury in experimental chronic kidney disease. Cell Commun. Signal. 2020, 18). A null biosensor was used to account for non-specific electro-active artefacts and subtracted from the ATP trace. Glycerol (2 mM) was included in all recording solutions to enable ATP detection. HK2 cells were seeded on glass coverslips (10 mm diameter) in low glucose DMEM/F12 (5 mmol/L) for 48 h, prior to an overnight serum-starvation. The cells were then incubated with TGFβ1 (10 ng/ml)±danegaptide (100 nM) for 48 h. The coverslips were transferred to a chamber containing Ca²⁺-containing BSS perfused at 6 ml/min (37° C.) and left for 10 min to acclimatise. ATP and null biosensors were bent and lowered so that the electrode laid parallel to the cellular monolayer. Once a stable baseline occurred, perfusion of Ca²⁺-free BSS stimulated hemichannel opening. After ATP release, Ca²⁺-containing BSS was used to close hemichannels, followed by a calibration solution of ATP (10 mM). Recordings were acquired at 4 Hz with a Micro CED (Mark2) interface using Spike (v8.03) software.

### Results

To determine if danegaptide (50-100 nM) could prevent TGFβ1 (10 ng/mL) induced release of ATP from hemichannels in hPTECs, we used ATP-biosensing. TGFβ1 (10 ng/mL) increased ATP release from 0.33±0.11 μM to 3.60±0.29 μM in hPTECs (FIGS. 2B & E; P≤0.001), an effect partially negated by danegaptide at both 50 nM (1.90±0.26 μM; P≤0.01) and 100 nM (0.79±0.19 μM; P≤0.001) (FIG. 2E, n=3, 6 repeats/sample number). Danegaptide alone did not affect ATP release (FIGS. 2C & E), with ATP levels recorded at 0.35±0.10 μM (50 nM) and 0.31±0.09 μM (100 nM) as compared to control (FIG. 2E).

### Conclusion

This example demonstrates that TGFβ1 (10 ng/mL) stimulates and stresses the renal epithelial cells to open their Cx43 based hemichannels allowing efflux of ATP to the extracellular matrix. Co-administration of danegaptide (both 50 nM and 100 nM) modified the Cx43 hemichannels to remain in a closed state despite TGFβ1-induced stress and thereby limited the leakage of ATP from the cytosol to the extracellular environment. Hence, danegaptide or a pharmaceutically acceptable salt thereof, emerges as a promising candidate for protecting renal epithelial cells from TGFβ1-induced stress and pathological hemichannel opening with resulting ATP leakage to the extracellular environment. It is well-known that high levels of ATP in the extracellular compartment is a potent stimulus for inflammation. Danegaptide hereby emerges as a relevant treatment of a kidney disease, which presents with inflammation and fibrosis development, and in particular Chronic Kidney Disease.

### Example 4—Danegaptide Reverses TGFβ1-Induced Changes in Cell Cycle Proteins and a Marker of Reno-Protection in hPTECs

### Materials and Methods

To determine if danegaptide can negate hemichannel-mediated regulation of common cell cycle and reno-protective markers, hPTECs were incubated with TGFβ1 (10 ng/mL)±danegaptide (100 nM) for 12 h and expression of candidate gene mRNA assessed through qPCR analysis.

RNA was extracted using an RNeasy mini kit (QIAGEN) and reverse transcribed (Invitrogen). Real-time PCR (SYBR GreenER, Invitrogen) was performed using a StepOne Plus instrument (Applied Biosystems Inc, Foster City, CA). cDNA expression was obtained by comparing to a standard curve of serially-diluted cDNA. The following primers were used: p16 (forward: CTCGTGCTGATGCTACTGAGGA, reverse: GGTCGGCGCAGTTGGGCTCC), p21 (forward: AGGTGGACCTGGAGACTCTCAG, reverse: TCCTCTTGGAGAAGATCAGCCG), cyclinD1 (forward: TCTACACCGACAACTCCATCCG, reverse: TCTGGCATTTTGGAGAGGAAGTG), Klotho (forward: CCTCCTTTACCTGAAAATCAGCC, reverse: CAGGTCGGTAAACTGAGACAGAG). Melt curve analysis confirmed primer specificity and checked for potential contamination.

### Results

Addition of danegaptide to TGFβ1-treated hPTECs returned expression of p16 from 358.8±21.1% to 193±18.5% of control, p21 from 221.8±12.9% to 142.2±6.2% and cyclinD1 from 253.2±7.7% to 132.9±15.0% (FIG. 3; 150 P≤0.001, n=3 respectively). In addition, danegaptide partially reversed the decline in Klotho from 43.3±3.8% to 59.9±11.7% of control, FIG. 3, n=3).

### Conclusion

This example demonstrates that TGFβ1-treated hPTECs responded with an increased expression of mRNA coding for cell cycle proteins such as p16, p21 and Cyclin D1 and that danegaptide was able to partly reduce the TGFβ1-induced changes of these mRNA levels. Further, this example demonstrates that TGFβ1 induced increased expression of p16, p21 and cyclin D1 and that these changes were restored when cells were co-incubated with danegaptide. Further, the data showed that TGFβ1 reduced the mRNA level of the important reno-protective factor called Klotho and that danegaptide was able to partially protect from this TGFβ1-induced decrease in Klotho. Reduced production of the Klotho protein has been observed in patients with chronic kidney failure and this may be one of the factors underlying the degenerative processes observed in CKD. Again, these data further support that danegaptide may have protective effects on the renal epithelial tissues exposed to the pro-fibrotic cytokine TGFβ1 and hence, danegaptide or a pharmaceutically acceptable salt thereof, emerges as a promising candidate for treatment of a kidney disease, and in particular Chronic Kidney Disease.

### Example 5—Danegaptide Restores TGFβ1-Mediated Changes in Adherens and Tight Junction Proteins and Paracellular Permeability in hPTECs

### Materials and Methods

To determine if danegaptide can negate TGFβ1-mediated changes in adherens and tight junction proteins, hPTECs were incubated with TGFβ1 (10 ng/mL)±danegaptide (100 nM) for 48 h and expression of candidate proteins assessed by Western blotting. Preparation of cytosolic protein from HK2 and HPTEC cells, their separation by SDS-gel electrophoresis and transfer onto Immobilon-FI PVDF membranes have been described previously (Price, G. W.; Chadjichristos, C. E.; Kavvadas, P.; Tang, S. C. W.; Yiu, W. H.; Green, C. R.; Potter, J. A.; Siamantouras, E.; Squires, P. E.; Hills, C. E. Blocking Connexin-43 mediated hemichannel activity protects against early tubular injury in experimental chronic kidney disease. Cell Commun. Signal. 2020, 18). Membranes were blocked using Odyssey blocking buffer (LI-COR), then probed overnight with antibodies against E-cadherin (1:1000), N-cadherin (1:1000), claudin-2 (1:500) and ZO-1 (1:1000), Col I (1:1000), Col IV (1:2000), Fibronectin (1:4000), Laminin (1:500), ILK1 (1:500), beta-catenin (1:2000), Vimentin (1:500), MMP3 (1:500). Bands were visualised using an OdysseyFC and semi-quantified using ImageStudio (v5.2, LI-COR). Additionally, transepithelial electrical resistance (TER) was measured as a functional measure of epithelial cell-cell coupling and paracellular permeability.

### Results

Danegaptide partially restored E-cadherin (ECAD) expression from 33.3±3.3% to 89±7.6% of control, N-cadherin (NCAD) from 224.4±29.6 to 161.9±27.4% and vimentin from 212.9±13% to 147.3±8.8% (FIG. 4A; P≤0.001, P≤0.01 and P≤0.001 respectively, n=3). Beta-catenin expression remained unaltered by danegaptide from 149.2±11.2% (TGFβ1 alone) to 151.6±16.8% (TGFβ1+danegaptide FIG. 4B; P=NS, n=3).

Assessment of the effects of danegaptide on tight junction proteins confirmed that the gap junction modifier partially restored expression of Claudin-2 from 44.5±5% to 74.6±5% of control and ZO-1 from 27.8±11.3% to 52.8±5.4%, as compared to control (FIG. 4B; P≤0.05, n=3). Studies examining transepithelial electrical resistance confirmed that reduced expression of tight junction proteins seen with TGFβ1 (10 ng/mL) was paralleled by a loss of transepithelial resistance from 57.33±1.86 Ω·cm2 to 10±1.53 Ω·cm2 (P≤0.001, n=3) This increased leakiness was partially corrected by co-incubation with danegaptide (36±2.08 Ω·cm2) (FIG. 4C; P≤0.001, n=3).

### Conclusion

This example demonstrates that danegaptide was able to maintain epithelial cell-cell coupling despite TGFβ1-induced stress. This protective effect of danegaptide was demonstrated both by expression of adherence and tight junction proteins and by functional measures of paracellular permeability. The loss of E-cadherin mediated cell adhesion and disassembly of tight junctions is considered to be an initiating trigger of partial Epithelial to Mesenchymal Transition, with disassembly of junctional proteins, a leaky epithelia and acquisition of mesenchymal proteins, such as vimentin and fibroblast specific protein all associated with the underlying pathology of tubulointerstitial fibrosis, severity of which dictates disease progression. Maintenance of cell-cell coupling and the ability of cells to communicate and synchronise their activity is known to be essential for the maintenance of tissue structure, integrity and function. Hence, danegaptide or a pharmaceutically acceptable salt thereof, emerges as a promising candidate for treatment of a kidney disease, and in particular Chronic Kidney Disease.

### Example 6—Danegaptide Prevents TGFβ1-Evoked Upregulation of Extracellular Matrix Proteins in hPTECs

### Materials and Methods

To examine the effect of danegaptide on TGFβ1-evoked changes in expression of ECM proteins, hPTEC cells were cultured in low (5 mM) glucose for 48 h, serum starved overnight and treated with TGFβ1 (10 ng/mL)±danegaptide (100 nM) for 48 h. Expression of the ECM proteins collagen I, collagen IV, fibronectin, Laminin, Integrin Linked Kinase 1, and Matrix Metalloproteinase 3 were assessed via western blotting.

### Results

Compared to control, TGFβ1 increased expression of the ECM proteins collagen I (334.6±30.14%), collagen IV (354.5±16.9%), fibronectin (301.7±50.4%) and laminin (324.8±36.4%) (FIGS. 5A & B; P≤0.001, n=3). Co-incubation with danegaptide significantly attenuated these changes, restoring expression to 180.7±27.3% (collagen I), 164.2±6.9% (collagen IV), 161.3±4% (fibronectin), 149±20.4% (laminin) (P≤0.001; n=3 in each case). Danegaptide (100 nM) also reduced TGFβ1 (10 ng/mL)-evoked changes in Integrin Linked Kinase 1 (ILK1) from 378.9±16.8% to 251.8±33% (P≤0.001), but had minimal effect on Matrix Metalloproteinase 3 (MMP3), reducing expression from 185.3±19.6% with the cytokine alone to 147.1±12.2% when co-incubated with danegaptide.

### Conclusion

This example demonstrates that TGFβ1 increased the expression of the ECM proteins known to be associated with the development of fibrosis, such as collagen I, collagen IV, fibronectin, and laminin. Moreover, Integrin-linked kinase is an intracellular serine/threonine protein kinase that plays a fundamental role in the regulation of cell adhesion, survival, proliferation, and extracellular matrix (ECM) deposition. Importantly, inhibition of ILK has been observed to attenuate renal fibrosis in multiple models of CKD. In the current study, TGFβ1-induced increases in ILK1 expression were restored when cells were co-incubated with danegaptide. Danegaptide was able to partially protect from TGFβ1-induced production of these ECM proteins. Hence, danegaptide or a pharmaceutically acceptable salt thereof, emerges as a promising candidate for treatment of a kidney disease, which presents with the development of fibrosis, and in particular Chronic Kidney Disease.

### Example 7—Danegaptide Reduces TGFβ1-Evoked Changes in Expression of Adipokines, Chemokines, Growth Factors and Interleukins from hPTECs

### Materials and Methods

A proteome profiler array (Human Cytokine Array Kit from R&D Systems, Oxfordshire, UK) was used, to determine if danegaptide negates TGFβ1-induced changes in expression and secretion of key pro-inflammatory mediators. Primary hPTECs were cultured as described above and treated with TGFβ1 (10 ng/mL)±danegaptide (100 nM) for 48 h.

### Results

A list of changes in lysate (FIG. 6, table 1) and supernatant (FIG. 7, table 2) are provided for 31 candidate proteins grouped by primary function.

Compared to control, TGFβ1 increased the expression of key cytokines and signal molecules of relevance in CKD and diabetic nephropathy including TNF-α, IFN-γ, IL-8, IGFBP-2, IGFBP-3 and ICAM-1. Co-incubation with danegaptide attenuated these changes, as a general pattern observed. Well-known and potent chemoattractants produced by activated tubular cells, such as MCP-1 (monocyte chemoattractant protein 1) and RANTES (Regulated on Activation Normal T-cells Expressed and Secreted chemokines) were also observed to be increased after TGFβ1 stimulation and these increases were observed to be attenuated when danegaptide was co-administered.

TABLE 1

| | | TGF-β1 (% compared | TGF-β1 + DG (% |
|---|---|---|---|
| Group | Protein | to control) | compared to control) |
| Adipokines | IFNg | 206 ± 15.4 | 112 ± 12.7 |
| | TNF-α | 192 ± 14.7 | 144 ± 18.5 |
| Cell function | Angiogenin | 237 ± 36 | 100 ± 20.3 |
| | Cystatin C | 182 ± 17.5 | 123 ± 14.3 |
| | Endoglin | 145 ± 24.9 | 72.3 ± 8.9 |
| | Kallikrein 3 | 76 ± 12.0 | 67 ± 14.3 |
| | MMP9 | 24 ± 3.1 | 95 ± 10.6 |
| | Serpin E1 | 186 ± 40.2 | 148 ± 28.5 |
| | Thrombospondin-1 | 128 ± 37.5 | 100 ± 19.8 |
| | TFF3 | 204 ± 25.8 | 132 ± 3.9 |
| Chemokines | ENA 78 | 207 ± 21.6 | 97 ± 18.2 |
| | ICAM 1 | 190 ± 13.6 | 80 ± 28.7 |
| | MCP-1 | 134 ± 30.2 | 97.6 ± 13.9 |
| | RANTES | 235 ± 13.5 | 104 ± 15.2 |
| Cytokines | G-CSF | 201 ± 24.6 | 126 ± 4.7 |
| | GM-CSF | 221 ± 11.1 | 136 ± 11.6 |
| | IGFBP2 | 519 ± 99.4 | 334 ± 66.1 |
| | IGFBP3 | 394 ± 94.5 | 97 ± 28.5 |
| | LIF | 328 ± 70.4 | 225 ± 82 |
| Interleukins | IL1A | 127 ± 26.4 | 80 ± 18.8 |
| | IL3 | 176 ± 3.4 | 129 ± 15.7 |
| | IL8 | 236 ± 9.4 | 122 ± 5.2 |
| | IL12 p70 | 177 ± 31.0 | 97 ± 4.6 |
| | IL13 | 269 ± 3.9 | 150 ± 46.0 |
| Growth Factors | FGF7 | 139 ± 5.6 | 106 ± 15.2 |
| | Flt 3 Ligand | 212 ± 13.3 | 208 ± 31.4 |
| | HGF | 153 ± 17.0 | 104.0 ± 9.0 |
| | PDGF-AA/BB | 247 ± 53.4 | 95.2 ± 14.6 |
| | VEGF | 203 ± 25.9 | 101 ± 16.4 |
| Receptors | DPPIV | 315 ± 6.8 | 187 ± 25.2 |
| | RAGE | 109 ± 23.8 | 105 ± 9.2 |

TABLE 2

| | | TGF-β1 (% compared | TGF-β1 + DG (% |
|---|---|---|---|
| Group | Protein | to control) | compared to control) |
| Adipokines | IFNg | 123 ± 11.0 | 90 ± 12.7 |
| | TNF-α | 176 ± 5.5 | 139 ± 6.1 |
| Cell function | Angiogenin | 184 ± 13.2 | 129 ± 14.7 |
| | Cystatin C | 169 ± 19.7 | 88 ± 6.8 |
| | Endoglin | 101 ± 10.6 | 94 ± 5.8 |
| | Kallikrein 3 | 75 ± 5.61 | 74 ± 20.6 |
| | MMP-9 | 41 ± 8.8 | 80 ± 8.9 |
| | Serpin E1 | 91 ± 4.7 | 81 ± 42.8 |
| | Thrombospondin-1 | 120 ± 24.8 | 115 ± 11.9 |
| | TFF3 | 114 ± 22.2 | 98 ± 17.5 |
| Chemokines | ENA 78 | 193 ± 17.7 | 119 ± 3.9 |
| | ICAM-1 | 191 ± 13.0 | 124 ± 16.6 |
| | MCP-1 | 171 ± 14.3 | 101 ± 6.2 |
| | RANTES | 140 ± 8.6 | 101 ± 12.1 |
| Cytokines | G-CSF | 171 ± 7.3 | 117 ± 12.0 |
| | GM-CSF | 213 ± 33.3 | 114 ± 3.8 |
| | IGFBP-2 | 259 ± 49.6 | 90 ± 6.4 |
| | IGFBP-3 | 147 ± 7.0 | 84.7 ± 14.3 |
| | LIF | 249 ± 32.1 | 134 ± 5.4 |
| Interleukins | IL1A | 109 ± 14.8 | 89 ± 21.5 |
| | IL3 | 127 ± 30.6 | 107 ± 38.0 |
| | IL8 | 138 ± 48.4 | 99 ± 26.4 |
| | IL12 p70 | 74 ± 11.5 | 63 ± 15.9 |
| | IL13 | 95 ± 16.4 | 62 ± 27.2 |
| Growth Factors | FGF7 | 125 ± 11.3 | 102 ± 16.5 |
| | Flt 3 Ligand | 179 ± 6.5 | 103 ± 12.8 |
| | HGF | 85 ± 15.8 | 88 ± 13.7 |
| | PDGF-AA/BB | 137 ± 9.7 | 99 ± 6.3 |
| | VEGF | 209 ± 43.3 | 217 ± 49.3 |
| Receptors | DPPIV | 222 ± 24.3 | 179 ± 37.8 |
| | RAGE | 170 ± 16.3 | 99 ± 12.5 |

### Conclusion

This example demonstrates that key cytokines, chemokines and other signal molecules known to be associated with inflammatory processes in several kidney diseases, including CKD and diabetic nephropathy were upregulated in response to TGFβ1 stimulation and that danegaptide demonstrated protective effects from these TGFβ1-induced changes in primary hPTECs. The inflammatory response in and around proximal tubules is known to involve both activation of multiple cell types combined with the secretion of numerous inflammatory markers. Specifically, soluble chemokines, cytokines and growth factors are known to recruit and activate infiltrating immune cells and stimulate resident fibroblasts. Sustained activation of these cells is associated with pathological inflammation and development of tubulointerstitial fibrosis. Hence, danegaptide or a pharmaceutically acceptable salt thereof, emerges as a promising candidate for treatment of a kidney disease, which presents with inflammation, and in particular Chronic Kidney Disease.

1. A compound of formula (I): 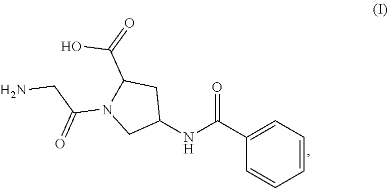 [SMILES: NCC(=O)N1CC(NC(=O)c2ccccc2)CC1C(=O)O]
or a pharmaceutically acceptable salt and/or hydrate thereof; for use in the treatment or prevention of a kidney disease, wherein the kidney disease is Chronic Kidney Disease (CKD) or an underlying condition leading to Chronic Kidney Disease (CKD).

2. The compound for use in the treatment or prevention according to claim 1, wherein the compound is administered to a human subject.

3. The compound for use in the treatment or prevention according to claim 2, wherein the subject is an infant subject, a child subject, an adolescent subject, an adult subject, or an elderly subject.

4. The compound for use in the treatment or prevention according to any one of the preceding claims, wherein the kidney disease presents in a subject with renal inflammation and/or development of renal fibrosis.

5. The compound for use in the treatment or prevention according to any one of the preceding claims, wherein the kidney disease is Chronic Kidney Disease (CKD).

6. The compound for use in the treatment or prevention according to any one of the preceding claims, wherein the underlying condition leading to Chronic Kidney Disease (CKD) is Diabetic Nephropathy that has resulted or may result in Chronic Kidney Disease (CKD).

7. The compound for use in the treatment or prevention according to any one of the preceding claims, wherein:
(a) the Chronic Kidney Disease is at stage 1, defined by kidney damage and GFR>90 ml/min/1.73 m² of the subject; or
(b) the Chronic Kidney Disease is at stage 2, defined by kidney damage and GFR 60-89 ml/min/1.73 m² of the subject; or
(c) the Chronic Kidney Disease is at stage 3a, defined by GFR 45-59 ml/min/1.73 m² of the subject; or
(d) the Chronic Kidney Disease is at stage 3b, defined by GFR 30-44 ml/min/1.73 m² of the subject; or
(e) the Chronic Kidney Disease is at stage 4, defined by GFR 15-29 ml/min/1.73 m² of the subject.

8. The compound for use in the treatment or prevention according to claim 7, wherein the compound prevents the progress from Chronic Kidney Disease at any one of stages 1, 2, 3a, 3b, or 4 to a more advanced Chronic Kidney Disease stage.

9. The compound for use in the treatment or prevention according to claim 7, wherein the compound prevents the progress from Chronic Kidney Disease at stage 1 to a Chronic Kidney Disease at any one of stages 2, 3a, 3b, 4, or 5.

10. The compound for use in the treatment or prevention according to claim 7, wherein the compound prevents the progress from Chronic Kidney Disease at stage 2 to a Chronic Kidney Disease at any one of stages 3a, 3b, 4, or 5.

11. The compound for use in the treatment or prevention according to claim 7, wherein the compound prevents the progress from Chronic Kidney Disease at stage 3a to a Chronic Kidney Disease at stage 3b, 4 or 5.

12. The compound for use in the treatment or prevention according to claim 7, wherein the compound prevents the progress from Chronic Kidney Disease at stage 3b to a Chronic Kidney Disease at stage 4 or 5.

13. The compound for use in the treatment or prevention according to claim 7, wherein the compound prevents the progress from Chronic Kidney Disease at stage 4 to a Chronic Kidney Disease at stage 5.

14. The compound for use in the treatment or prevention according to any one of the preceding claims, wherein the kidney disease presents with proteinuria, albuminuria or micro-albuminuria.

15. The compound for use in the treatment or prevention according to any one of the preceding claims, wherein the underlying condition leading to Chronic Kidney Disease (CKD) is a glomerular disease, such as glomerulosclerosis or diabetic glomerulosclerosis.

16. The compound for use in the treatment or prevention according to any one of the preceding claims, wherein the underlying condition leading to Chronic Kidney Disease (CKD) selected from the group consisting of: Systemic lupus erythematosus (lupus nephritis), Diabetic nephropathy, Sarcoidosis, Sjögren's syndrome, Amyloidosis, Multiple myeloma, Vasculitis, Atresia or Stenosis of the Ureter, Calculus (kidney stone) of Upper or Lower urinary tract, or Obstructive and Reflux Nephropathy.

17. The compound for use in the treatment or prevention according to any one of the preceding claims, wherein the underlying condition leading to Chronic Kidney Disease (CKD) is Renal Tubulo-Interstitial Disease.

18. The compound for use in the treatment or prevention according to claim 17, wherein the Renal Tubulo-Interstitial Disease is selected from Chronic Tubulo-Interstitial Nephritis, Drug- and Heavy-Metal-Induced Tubulo-Interstitial and Tubular Conditions and Renal Tubulo-Interstitial Disorders in Systemic Connective Tissue Disorders.

19. The compound for use in the treatment or prevention according to any one of the preceding claims, wherein the underlying condition leading to Chronic Kidney Disease (CKD) is a rare inherited kidney disease.

20. The compound for use in the treatment or prevention according claim 19, wherein the rare inherited kidney disease is polycystic kidney disease (PKD).

21. The compound for use in the treatment or prevention according to any one of the preceding claims, wherein the compound inhibits hemi-channel ATP release.

22. The compound for use in the treatment or prevention according to claim 21, wherein the compound inhibits Cx43 mediated ATP release.

23. The compound for use in the treatment or prevention according to any one of the preceding claims, wherein:
(a) the compound restores changes in expression and secretion of extracellular matrix proteins, adipokines, chemokines and growth factors induced by TGFβ1;
and/or
(b) the compound protects against changes in renal function associated with inflammation and fibrosis; and/or
(c) the compound decreases inflammation in the proximal tubule or tubulointestinal tissues.

24. The compound for use in the treatment or prevention according to any one of the preceding claims, wherein the compound is represented by the formula: 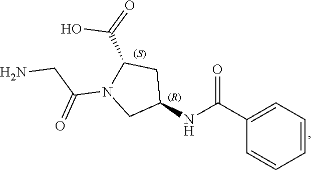
or a pharmaceutically acceptable salt thereof.

25. The compound for use in the treatment or prevention according to any one of the preceding claims, wherein the use provides for a target concentration of the administered compound of about 50 nM to about 100 nM in the microenvironment of the renal tissues.

26. The compound for use in the treatment or prevention according to any one of the preceding claims, wherein the compound is administered in the range of about 10 mg to about 500 mg per subject per day.

27. The compound for use in the treatment or prevention according to claim 26, wherein the compound is administered in the range of about 10 mg to about 100 mg per subject per day.

28. The compound for use in the treatment or prevention according to claim 26, wherein the compound is administered in the range of about 50 mg to about 150 mg per subject per day.

29. The compound for use in the treatment or prevention according to any one of the preceding claims, wherein the compound is administered in at least about 0.1 mg/kg body weight of subject per day.

30. The compound for use in the treatment or prevention according to any one of the preceding claims, wherein the compound is administered in at least about 0.3 mg/kg body weight of subject per day.

31. The compound for use in the treatment or prevention according to any one of the preceding claims, wherein the compound is administered in at least about 0.5 mg/body weight of subject per day.

32. The compound for use in the treatment or prevention according to any one of the preceding claims, wherein the compound is administered in at least about 1 mg/kg body weight of subject per day.

33. The compound for use in the treatment or prevention according to any one of the preceding claims, wherein the compound is administered in at least about 2 mg/body weight of subject per day.

34. The compound for use in the treatment or prevention according to any one of the preceding claims, wherein the pharmaceutically acceptable salt is a hydrochloride salt.

35. The compound for use in the treatment or prevention according to any one of the preceding claims, wherein the compound is administered orally or parenterally.

36. The compound for use in the treatment or prevention according to any one of the preceding claims, wherein the compound is administered orally.

37. The compound for use in the treatment or prevention according to any one of the preceding claims, wherein the compound is administered once or twice daily.

38. A method for treatment or prevention of a kidney disease in a subject, wherein the kidney disease is Chronic Kidney Disease (CKD) or an underlying condition leading to Chronic Kidney Disease (CKD), wherein the method comprises administering a compound of formula (I): 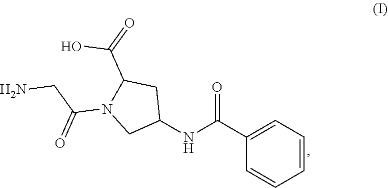 [SMILES: NCC(=O)N1CC(NC(=O)c2ccccc2)CC1C(=O)O]
or a pharmaceutically acceptable salt and/or hydrate thereof, to the subject.

39. Use of a compound of formula (I): 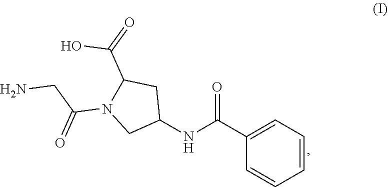 [SMILES: NCC(=O)N1CC(NC(=O)c2ccccc2)CC1C(=O)O]
or a pharmaceutically acceptable salt and/or hydrate thereof, for the manufacture of a medicament for the treatment or prevention of a kidney disease, wherein the kidney disease is Chronic Kidney Disease (CKD) or an underlying condition leading to Chronic Kidney Disease (CKD).

40. A method for (a) restoring changes in expression and secretion of extracellular matrix proteins, adipokines, chemokines and growth factors induced by TGFβ1; and/or (b) protecting against changes in renal function associated with inflammation and fibrosis; and/or (c) decreasing inflammation in the proximal tubule or tubulointestinal tissues, in a subject, said method comprising administering a compound of formula (I): 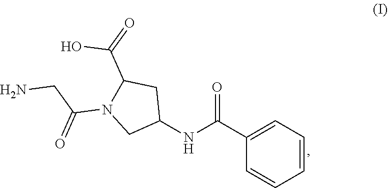 [SMILES: NCC(=O)N1CC(NC(=O)c2ccccc2)CC1C(=O)O]
or a pharmaceutically acceptable salt and/or hydrate thereof, to the subject.

41. A method for selecting a subject for treatment by a compound of formula (I): 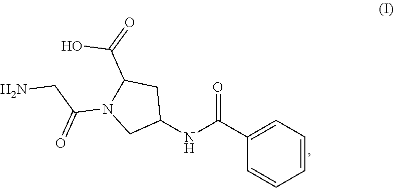 [SMILES: NCC(=O)N1CC(NC(=O)c2ccccc2)CC1C(=O)O]
or a pharmaceutically acceptable salt and/or hydrate thereof, wherein the method comprises:
(a) providing a sample or a biopsy from the subject;
(b) determining the presence of one or more kidney disease biomarkers in the sample or biopsy;
(c) selecting the subject for treatment if one or more kidney disease biomarkers in the sample or biopsy is present.

42. A method for selecting a subject for treatment by a compound of formula (I): 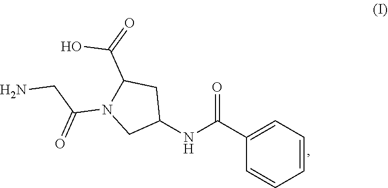 [SMILES: NCC(=O)N1CC(NC(=O)c2ccccc2)CC1C(=O)O]
or a pharmaceutically acceptable salt and/or hydrate thereof, wherein the method comprises:
(a) determining the presence of one or more kidney disease biomarkers in a sample or biopsy obtained from a patient;
(b) selecting the subject for treatment if one or more kidney disease biomarkers in the sample or biopsy is present.

43. The method for selecting a subject for treatment of claim 41 or 42, wherein the one or more kidney disease biomarkers is selected from proteinuria, GFR estimations, urine albumin-to-creatinine ratio and/or biomarkers of inflammation or fibrosis development.

44. A method for selecting a subject for treatment by a compound of formula (I): 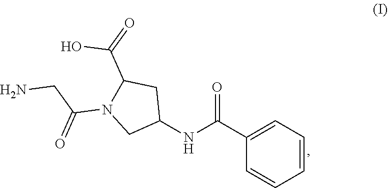 [SMILES: NCC(=O)N1CC(NC(=O)c2ccccc2)CC1C(=O)O]
or a pharmaceutically acceptable salt and/or hydrate thereof, wherein the method comprises:
(a) providing a sample or a biopsy from the subject;
(b) determining thickening of basal membrane (BM) and/or mesangial matrix expansion, relative to a baseline level from a control sample;
wherein a thickened basal membrane and/or an expanded mesangial matrix indicates that the subject is responsive to treatment with the compound.

45. A method for selecting a subject for treatment by a compound of formula (I): 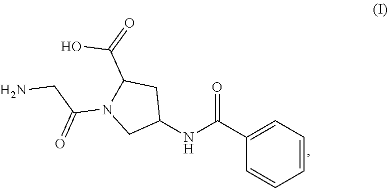 [SMILES: NCC(=O)N1CC(NC(=O)c2ccccc2)CC1C(=O)O]
or a pharmaceutically acceptable salt and/or hydrate thereof, wherein the method comprises:
(a) determining thickening of basal membrane (BM) and/or mesangial matrix expansion in a sample or biopsy obtained from a patient, relative to a baseline level from a control sample;
wherein a thickened basal membrane and/or an expanded mesangial matrix indicates that the subject is responsive to treatment with the compound.

46. The method for selecting a subject for treatment of any one of claims 41-45, wherein the method is ex vivo.

47. The method for selecting a subject for treatment of any one of claims 41-45, wherein the method is in vitro.

48. A method for treatment or prevention of a kidney disease in a subject, wherein the kidney disease is Chronic Kidney Disease (CKD) or an underlying condition leading to Chronic Kidney Disease (CKD), wherein the method comprises administering a compound of formula (I): 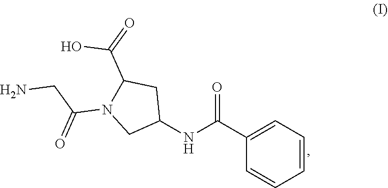 [SMILES: NCC(=O)N1CC(NC(=O)c2ccccc2)CC1C(=O)O]
or a pharmaceutically acceptable salt and/or hydrate thereof, to the subject, wherein the subject is selected according to the method for selecting a subject for treatment according to any one of claims 41-47.
